(19) United States
(12) Patent Application Publication
(10) Pub. No.: US 2022/0275337 A1
(43) Pub. Date: Sep. 1, 2022

(54) CELLULAR COMPOSITIONS COMPRISING VIRAL VECTORS AND METHODS OF TREATMENT

(71) Applicant: Mesoblast International Sárl, Meyrin (CH)

(72) Inventors: Dan DEVINE, New York, NY (US); Nick LOIZOS, New York, NY (US); Silviu ITESCU, Melbourne (AU)

(21) Appl. No.: 17/633,063

(22) PCT Filed: Aug. 5, 2020
(86) PCT No.: PCT/IB2020/057410

§ 371 Date: Feb. 4, 2022

Related U.S. Application Data
(60) Provisional application No. 62/882,840, filed on Aug. 5, 2019.

(51) Int. Cl. C12N 5/0775; A61K 35/28; A61P 35/00; C12N 15/86

(52) U.S. Cl. CPC C12N 5/0662; A61K 35/28; A61P 35/00; C12N 15/86

(57) ABSTRACT

The present disclosure relates to cellular compositions that are modified to introduce a recombinant virus. Such compositions may be used to treat cancer by delivering vims to cancer cells.

### CROSS REFERENCE TO RELATED APPLICATIONS

The application claims the benefit of U.S. Provisional Application No. 62/882,840, filed Aug. 5, 2019, which is hereby incorporated by reference in its entirety.

### REFERENCE TO A SEQUENCE LISTING SUBMITTED ELECTRONICALLY VIA EFS-WEB

The content of the electronically submitted sequence listing (Name: 3944_063PC01_SL_ST25.txt; Size: 6,885 bytes; and Date of Creation: Aug. 3, 2020) is hereby incorporated by reference pursuant to 37 C.F.R. § 1.52(e)(5).

### FIELD OF THE INVENTION

The present disclosure relates to cellular compositions that are modified to introduce a recombinant virus. Such compositions may be used to treat cancer by delivering virus to cancer cells.

### BACKGROUND OF THE INVENTION

Treatment of cancer typically involves surgical resection, standard chemotherapy and/or radiation therapy to remove or kill cancer cells. However, the effectiveness of these treatments is often limited because of the invasiveness of the tumour and/or collateral damage to healthy tissues. This situation signifies a need for novel therapeutic strategies, and one such approach is the use of viruses.

Oncolytic viruses are viruses that are able to replicate specifically in and destroy cancer cells, and this property is either inherent or genetically-engineered. Unfortunately, promising laboratory results are yet to be translated into improved clinical outcomes, and this appears to be determined by the complex interactions between the tumour and its microenvironment, the virus, and the host immunity.

Accordingly, improved compositions and methods of delivering viruses to tumour cells are required.

### SUMMARY OF THE INVENTION

The present inventors have identified that mesenchymal lineage precursor or stem cells can be modified to enhance killing of tumour cells. For example, the present inventors have identified that modified mesenchymal lineage precursor or stem cells can deliver payload to tumour cells to reduce tumour cell growth. The present inventors have also identified modifications that can enhance migration of modified mesenchymal lineage precursor or stem cells to tumour cells. In an example, the present inventors have identified that increasing expression of Phosphatase and Tensin Homolog deleted on chromosome 10 alpha (PTENα) in mesenchymal lineage precursor or stem cells can enhance the ability of these cells to migrate to and/or kill tumour cells. These findings suggest that modified cells according to the present disclosure can advantageously home to tumour cells and deliver therapeutic payload.

Accordingly, in a first aspect, the present disclosure encompasses a population of mesenchymal lineage precursor or stem cells, wherein said cells are modified to increase expression of Phosphatase and Tensin Homolog deleted on chromosome 10 alpha (PTENα). In an example, the increase in expression of PTENα is sufficient to decrease the level of phosphorylated AKT in modified cells. In another example, the increase in expression of PTENα is sufficient to enhance killing of tumour cells. In another example, the increase in expression of PTENα is sufficient to enhance migration to tumour cells. In another example, the increase in expression of PTENα is sufficient to enhance both migration to tumour cells and killing of tumour cells.

In another example, the mesenchymal lineage precursor or stem cells are modified to introduce a recombinant virus. For example, the mesenchymal lineage precursor or stem cells may be modified to introduce a recombinant virus comprising a herpes simplex virus (HSV) backbone.

In an example, the mesenchymal lineage precursor or stem cells are modified to introduce a recombinant virus which comprises a polynucleotide encoding PTENα. In an example, the recombinant virus is an oncolytic virus.

The present inventors have also identified mesenchymal lineage precursor or stem cells as an effective carrier of recombinant virus comprising a herpes simplex virus (HSV) backbone and expressing a PTEN transgene noting a particularly high rate of infectivity and replication with these viral constructs. Taken together with the above noted capabilities of modified cells according to the present disclosure, the inventor findings suggest that mesenchymal lineage precursor or stem cells comprising modifications discussed herein may represent novel and effective compositions for treating various cancers that are commercially scalable, particularly when modified to introduce a recombinant virus comprising a HSV backbone. Accordingly, in an example the recombinant virus comprises a herpes simplex virus (HSV) backbone.

In an example, the HSV has a high rate of infectivity of mesenchymal lineage precursor or stem cells. In an example, at least 10% of cells in a population disclosed herein comprise virus. In another example, at least 20% of cells in a population disclosed herein comprise virus. In another example, between 20% and 80% of cells in a population disclosed herein comprise virus.

In an example, the polynucleotide encoding PTEN-alpha is operatively linked to a tumour specific promoter. In a further example, the tumour specific promoter is a survivin promoter, COX-2 promoter, PSA promoter, CXCR4 promoter, STAT3 promoter, hTERT promoter, AFP promoter, CCKAR promoter, CEA promoter, erbB2 promoter, E2F1 promoter, HE4 promoter, LP promoter, MUC-1 promoter, TRP1 promoter, Tyr promoter.

In an example, the polynucleotide encoding PTEN-alpha is operatively linked to an inducible promoter.

In an example, the recombinant virus comprises a capsid protein that binds a tumour-specific cell surface molecule. In a further example, the capsid protein is a fibre, a penton or hexon protein.

In an example, the recombinant virus comprises a nucleic acid sequence as shown in SEQ ID NO: 1 or a variant thereof that is translated into a protein comprising an amino acid sequence as shown in SEQ ID NO: 2. In an example, the variant of SEQ ID NO: 1 shares at least 85%, 90%, 95%, 99% sequence identity with SEQ ID NO: 2.

In an example, the recombinant virus is a HSV.

In an example, the tumour cells are breast cancer or brain cancer cells.

In an example, the mesenchymal lineage precursor or stem cell expresses one or more of the markers selected from the group consisting of α1, α2, α3, α4 and α5, αv, β1 and β3. In an example, the mesenchymal lineage precursor or stem cell expresses STRO-1. In another example, the mesenchymal lineage precursor or stem cells are substantially STRO-1^{bri}. In an example, the mesenchymal lineage precursor or stem cells express Angl:VEGF at a ratio of at least 2:1 to 30:1. In another example, the mesenchymal lineage precursor or stem cells express Angl:VEGF at a ratio of at least about 10:1. In a further example, the mesenchymal lineage precursor or stem cells express Ang1:VEGF at a ratio of at least about 20:1. In another example, the mesenchymal lineage precursor or stem cells express Angl:VEGF at a ratio of at least about 30:1.

In an example, the mesenchymal lineage precursor or stem cells are not genetically modified to express Ang1 or VEGF. In an example, the mesenchymal lineage precursor or stem cells are derived from pluripotent cells. In another example, the pluripotent cells are induced pluripotent stem (iPS) cells.

In an example, the mesenchymal lineage precursor or stem cells express STRO-1 and two or more of the markers selected from the group consisting of α1, α2, α3, α4 and α5, αv, β1 and β3.

In an example, the population of cells has been culture expanded.

In an example, there is provided a pharmaceutical composition comprising a population disclosed herein.

The present inventors have also identified that they are able to increase PTENα expression in a cell by contacting the cell with a population of modified mesenchymal lineage precursor or stem cells disclosed herein. In an example, the contacted cell is a cancer cell. In an example, the increase in PTENα expression in the contacted cell reduces the level of phosphorylated AKT in the cell.

The present disclosure also encompasses a method of treating cancer in a subject, the method comprising administering a population or composition according to any one of the examples provided above. In an example, the present disclosure also encompasses a method of killing cancer cells, the method comprising contacting a population of cancer cells with a population or composition according to any one of the examples provided above. In another example, the present disclosure also encompasses a method of delivering mesenchymal lineage precursor or stem cells to cancer cells in a subject, the method comprising administering a population or composition of any one of the examples provided above. In an example, the cancer is selected from the group consisting of lung cancer, pancreatic cancer, colorectal cancer, liver cancer, cervical cancer, prostate cancer, breast cancer, endometrial cancer, thyroid cancer, kidney cancer, brain cancer, glioblastoma, osteosarcoma and melanoma. In a further example, the cancer is breast cancer or brain cancer. In another example, the population or composition is administered to the subject by intravenous, intra-arterial or intraperitoneal administration. In an example, the composition is administered directly into a subjects tumour.

In another example, the present disclosure relates to use of a population disclosed herein in the manufacture of a medicament for treating cancer. In another example, the present disclosure relates to Use of a population disclosed herein in the manufacture of a medicament for delivering mesenchymal lineage precursor or stem cells to cancer cells.

Any example herein shall be taken to apply mutatis mutandis to any other example unless specifically stated otherwise.

The present disclosure is not to be limited in scope by the specific examples described herein, which are intended for the purpose of exemplification only. Functionally-equivalent products, compositions and methods are clearly within the scope of the disclosure, as described herein.

Throughout this specification, unless specifically stated otherwise or the context requires otherwise, reference to a single step, composition of matter, group of steps or group of compositions of matter shall be taken to encompass one and a plurality (i.e. one or more) of those steps, compositions of matter, groups of steps or group of compositions of matter.

The disclosure is hereinafter described by way of the following non-limiting

Examples and with reference to the accompanying drawings.

### BRIEF DESCRIPTION OF THE ACCOMPANYING DRAWINGS

FIG. 1 (A and B). Viral backbone of HSVQ (parental virus) and HSV-P10 (PTENα expressing virus).

FIG. 2 (A and B). HSV-P10 loading of mesenchymal stem cells (MSC).

FIG. 3 (A and B). Viability of HSV-P10 and HSVQ loaded mesenchymal stem cells (MSC).

FIG. 4 (A and B). Expression of PTENα of HSV-P10 loaded mesenchymal stem cells (MSC) and effects on PI3K/AKT signalling pathway.

FIG. 5. Migration of HSV-P10 and HSVQ loaded mesenchymal stem cells (MSC) towards human breast cancer cells (MDA-468).

FIG. 6 (A and B). Effect of HSV-P10 loaded mesenchymal stem cells (MSC) on human glioma cells.

FIG. 7. Induction of tumour cell death of DB7 murine breast cancers cells co-cultured with HSV-P10 and HSVQ loaded mesenchymal stem cells (MSC).

### DETAILED DESCRIPTION OF THE INVENTION

### General Techniques and Selected Definitions

Unless specifically defined otherwise, all technical and scientific terms used herein shall be taken to have the same meaning as commonly understood by one of ordinary skill in the art (e.g., molecular biology, cell culture, stem cell differentiation, cell therapy, genetic modification, virology, oncology, biochemistry, physiology, and clinical studies).

Unless otherwise indicated, the molecular and statistical techniques utilized in the present disclosure are standard procedures, well known to those skilled in the art. Such techniques are described and explained throughout the literature in sources such as, J. Perbal, A Practical Guide to Molecular Cloning, John Wiley and Sons (1984), J. Sambrook et al., Molecular Cloning: A Laboratory Manual, Cold Spring Harbour Laboratory Press (1989), T.A. Brown (editor), Essential Molecular Biology: A Practical Approach, Volumes 1 and 2, IRL Press (1991), D.M. Glover and B.D. Hames (editors), DNA Cloning: A Practical Approach, Volumes 1-4, IRL Press (1995 and 1996), and F. M. Ausubel et al. (editors), Current Protocols in Molecular Biology, Greene Pub. Associates and Wiley-Interscience (1988, including all updates until present), Ed Harlow and David Lane (editors) Antibodies: A Laboratory Manual, Cold Spring Harbour Laboratory, (1988), and J. E. Coligan et al. (editors) Current Protocols in Immunology, John Wiley & Sons (including all updates until present).

As used in this specification and the appended claims, terms in the singular and the singular forms “a,” “an” and “the,” for example, optionally include plural referents unless the content clearly dictates otherwise. Thus, for example, reference to “an analyte” optionally includes one or more analytes.

As used herein, the term “about”, unless stated to the contrary, refers to +/−10%, more preferably +/−5%, more preferably +/−1%, of the designated value.

The term “and/or”, e.g., “X and/or Y” shall be understood to mean either “X and Y” or “X or Y” and shall be taken to provide explicit support for both meanings or for either meaning.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

The term “phosphatase and tensin homolog deleted on chromosome 10 (PTEN)” is used in the context of the present disclosure to refer to the gene which encodes phosphatidylinositol-3,4,5-trisphosphate 3-phosphatase (PTEN; Gene ID: 5728; UniProtKB# P60484). The term “PTEN-alpha” or “PTEN-α” is used to refer to a 576-amino acid translational variant of PTEN (P60484-2; a.k.a PTEN-Long), that arises from an alternative translation start site 519 base pairs upstream of the ATG initiation sequence, adding 173 N-terminal amino acids to the normal PTEN open reading frame. In an example, PTEN-alpha comprises an amino acid sequence as shown in SEQ ID NO: 2. In another example, PTEN-alpha is as described in Hopkins et al. (2013) Science., 6144:399-402. The term, “phosphatase and tensin homolog deleted on chromosome 10 (PTEN) alpha” is used in the context of the present disclosure to refer to the gene which encodes PTEN-alpha. In an example, PTEN-alpha is encoded by a nucleic acid comprising SEQ ID NO: 1 or a variant thereof that encodes a protein comprising an amino acid sequence as shown in SEQ ID NO: 2. Accordingly, in an example, a population of cells disclosed herein can be modified to increase expression of a nucleic acid that encodes a protein comprising an amino acid sequence as shown in SEQ ID NO: 2. In an example, the nucleic acid expressing PTEN-alpha is modified to facilitate higher levels of PTEN-alpha translation than PTEN translation. In an example, the nucleic acid expressing PTEN-alpha is not translated into PTEN in a cancer cell.

In another example, PTEN-alpha is encoded by a nucleic acid comprising a sequence corresponding to the PTEN gene (Gene ID: 5728) wherein the PTEN-alpha CUG start codon is mutated to AUG. In this example, the PTEN AUG start codon can also be mutated. For example, the PTEN AUG start codon is mutated to AUA. In these examples, a population of cells disclosed herein can be modified to increase expression of such nucleic acids.

As used herein, a “PTEN mutated or deficient cancer” is a cancer which has been identified by testing a sample of the cancer from an individual to have one or more mutations in a PTEN protein or where the PTEN gene is absent or reduced compared to level of the protein/gene in normal cells. PTEN mutation or deficiency has been observed in a number of cancers including glioblastoma, endometrial cancer, colon cancer, lung cancer, breast cancer, prostate cancer and ovarian cancer. In an example, the PTEN mutated or deficient cancer has a mutation in PTEN. In another example, PTEN mutated or deficient cancer has a mutation in PTEN-alpha.

Various subjects can be administered cell compositions according to the present disclosure. In an example, the subject is a mammal. The mammal may be a companion animal such as a dog or cat, or a livestock animal such as a horse or cow. In another example, the subject is a human. Terms such as “subject”, “patient” or “individual” are terms that can, in context, be used interchangeably in the present disclosure.

As used herein, the term “treatment” refers to clinical intervention designed to alter the natural course of the individual or cell being treated during the course of clinical pathology. Desirable effects of treatment include decreasing the rate of disease progression, ameliorating or palliating the disease state, and remission or improved prognosis. An individual is successfully “treated”, for example, if one or more symptoms associated with a disease are mitigated or eliminated.

An “effective amount” refers to at least an amount effective, at dosages and for periods of time necessary, to achieve the desired therapeutic or prophylactic result. An effective amount can be provided in one or more administrations. In some examples of the present disclosure, the term “effective amount” is used to refer to an amount necessary to effect treatment of a disease or condition as hereinbefore described. The effective amount may vary according to the disease or condition to be treated and also according to the weight, age, racial background, sex, health and/or physical condition and other factors relevant to the mammal being treated. Typically, the effective amount will fall within a relatively broad range (e.g. a “dosage” range) that can be determined through routine trial and experimentation by a medical practitioner. The effective amount can be administered in a single dose or in a dose repeated once or several times over a treatment period.

A “therapeutically effective amount” is at least the minimum concentration required to effect a measurable improvement of a particular disorder (e.g. cancer). A therapeutically effective amount herein may vary according to factors such as the disease state, age, sex, and weight of the patient, and the ability of the cellular composition to elicit a desired response in the individual. A therapeutically effective amount is also one in which any toxic or detrimental effects of the composition are outweighed by the therapeutically beneficial effects. In the case of cancer, a therapeutically effective amount can reduce the number of cancer cells; reduce the primary tumour size; inhibit (i.e., slow to some extent and, in some examples, stop) cancer cell infiltration into peripheral organs; inhibit (i.e., slow to some extent and, in some examples, stop) tumour metastasis; inhibit or delay, to some extent, tumour growth or tumour progression; and/or relieve to some extent one or more of the symptoms associated with the disorder. To the extent a composition according to the present disclosure may prevent growth and/or kill existing cancer cells, it may be cytostatic and/or cytotoxic. For cancer therapy, efficacy in vivo can, for example, be measured by assessing the duration of survival, time to disease progression (TTP), the response rates (RR), duration of response, and/or quality of life.

In an example, the level of a particular marker is determined under culture conditions. The term “culture conditions” is used to refer to cells growing in culture. In an example, culture conditions refers to an actively dividing population of cells. Such cells may, in an example, in exponential growth phase. For example, the level of a particular marker can be determined by taking a sample of cell culture media and measuring the level of marker in the sample. In another example, the level of a particular marker can be determined by taking a sample of cells and measuring the level of the marker in the cell lysate. Those of skill in the art that secreted markers will be measured by sampling the culture media while markers expressed on the surface of the cell may be measured by assessing a sample of cell lysate. In an example, the sample is taken when the cells are in exponential growth phase. In an example, the sample is taken after at least two days in culture.

Culture expanding cells from a cryopreserved intermediate means thawing cells subject to cryogenic freezing and in vitro culturing under conditions suitable for growth of the cells.

### Mesenchymal Lineage Precursor or Stem Cells

As used herein, the term “mesenchymal lineage precursor or stem cells” refers to undifferentiated multipotent cells that have the capacity to self renew while maintaining multipotentcy and the capacity to differentiate into a number of cell types either of mesenchymal origin, for example, osteoblasts, chondrocytes, adipocytes, stromal cells, fibroblasts and tendons, or non-mesodermal origin, for example, hepatocytes, neural cells and epithelial cells. In various examples, the present disclosure encompasses a population of mesenchymal lineage precursor or stem cells, wherein said cells are modified to enhance migration to tumour cells. In an example, the present disclosure encompasses a population of mesenchymal lineage precursor or stem cells comprising a recombinant virus, wherein said cells are modified to enhance migration to tumour cells. For example, the present disclosure encompasses a population of mesenchymal lineage precursor or stem cells, wherein said cells are modified to increase expression of Phosphatase and Tensin Homolog deleted on chromosome 10 alpha (PTENα) sufficient to enhance migration to the tumour cells. In another example, the present disclosure encompasses a population of mesenchymal lineage precursor or stem cells, wherein said cells are modified to introduce a recombinant virus which comprises a polynucleotide encoding PTENα. In this example, expression of PTENα from the virus and translation of the same to PTENα protein is sufficient to enhance migration to tumour cells.

The term “mesenchymal lineage precursor or stem cells” includes both parent cells and their undifferentiated progeny. The term also includes mesenchymal lineage precursor or stem cells (MPC), multipotent stromal cells, mesenchymal stem cells, perivascular mesenchymal lineage precursor or stem cells, and their undifferentiated progeny. Accordingly, in an example, the mesenchymal lineage precursor or stem cells are mesenchymal stem cells.

Mesenchymal lineage precursor or stem cells can be autologous, allogeneic, xenogeneic, syngeneic or isogeneic. Autologous cells are isolated from the same individual to which they will be reimplanted. Allogeneic cells are isolated from a donor of the same species. Xenogeneic cells are isolated from a donor of another species. Syngeneic or isogeneic cells are isolated from genetically identical organisms, such as twins, clones, or highly inbred research animal models.

In an example, the mesenchymal lineage precursor or stem cells are allogeneic. In an example, the allogeneic mesenchymal lineage precursor or stem cells are culture expanded and cryopreserved.

Mesenchymal lineage precursor or stem cells reside primarily in the bone marrow, but have also been shown to be present in diverse host tissues including, for example, cord blood and umbilical cord, adult peripheral blood, adipose tissue, trabecular bone and dental pulp.

In an example, mesenchymal lineage precursor or stem cells express STRO-1 and one or more integrins. Integrins are a class of cell adhesion receptors that mediate both cell-cell and cell-extracellular matrix adhesion events. Integrins consist of heterodimeric polypeptides where a single a chain polypeptide noncovalently associates with a single β chain. There are now about 16 distinct a chain polypeptides and at least about 8 different β chain polypeptides that constitute the integrin family of cell adhesion receptors. In general, different binding specificities and tissue distributions are derived from unique combinations of the α and β chain polypeptides or integrin subunits. The family to which a particular integrin is associated with is usually characterized by the β subunit. However, the ligand binding activity of the integrin is largely influenced by the a subunit.

In an example, mesenchymal lineage precursor or stem cells according to the present disclosure express STRO-1 and an integrin having a β1 (CD29) chain polypeptide.

In another example, mesenchymal lineage precursor or stem cells according to the present disclosure express STRO-1 and an integrin having an a chain polypeptide selected from the group consisting of α1 (CD49a), α2 (CD49b), α3 (CD49c), α4 (CD49d), α5 (CD49e) and αv (CD51). Accordingly, in an example, mesenchymal lineage precursor or stem cells according to the present disclosure express STRO-1 and al. In another example, mesenchymal lineage precursor or stem cells express STRO-1 and α2. In another example, mesenchymal lineage precursor or stem cells express STRO-1 and α3. In another example, mesenchymal lineage precursor or stem cells express STRO-1 and α4. In another example, mesenchymal lineage precursor or stem cells express STRO-1 and α5. In another example, mesenchymal lineage precursor or stem cells express STRO-1 and αv. In another example, mesenchymal lineage precursor or stem cells express STRO-1, α2 and α3. In another example, mesenchymal lineage precursor or stem cells express STRO-1, α2 and α5. In another example, mesenchymal lineage precursor or stem cells express STRO-1, α3 and α5. In another example, mesenchymal lineage precursor or stem cells express STRO-1, α2, α3 and α5.

In another example, the present disclosure encompasses a population of mesenchymal lineage precursor or stem cells enriched for STRO-1 and al+cells. In this example, a population enriched for al+cells can comprise at least about 3% or 4% or 5% α1+cells.

In another example, the present disclosure encompasses a population of mesenchymal lineage precursor or stem cells enriched for STRO-1 and α2+cells. In this example, a population enriched for α2+cells can comprise at least about 30% or 40% or 50% α2+cells.

In another example, the present disclosure encompasses a population of mesenchymal lineage precursor or stem cells enriched for STRO-1 and α3+cells. In this example, a population enriched for α3+cells comprises at least about 40% or 45% or 50% α3+cells.

In another example, the present disclosure encompasses a population of mesenchymal lineage precursor or stem cells enriched for STRO-1 and α4+cells. In this example, a population enriched for α4+cells comprises at least about 5% or 6% or 7% a4+cells.

In another example, the present disclosure encompasses a population of mesenchymal lineage precursor or stem cells enriched for STRO-1 and α5+cells. In this example, a population enriched for α5+cells comprises at least about 45% or 50% or 55% α5+cells.

In another example, the present disclosure encompasses a population of mesenchymal lineage precursor or stem cells enriched for STRO-1 and av+cells. In this example, a population enriched for av+cells comprises at least about 5% or 6% or 7% av+cells.

In another example, the present disclosure encompasses a population of mesenchymal lineage precursor or stem cells enriched for STRO-1, al+, α3+, α4+and a5+cells.

In the above examples, the mesenchymal lineage precursor or stem cell can have a pi chain polypeptide. For example, mesenchymal lineage precursor or stem cells according to the present disclosure can express an integrin selected from the group consisting of al(31, α2(31, α3(31, α4(31 and α5(31. Accordingly, in an example, mesenchymal lineage precursor or stem cells according to the present disclosure express STRO-1 and al(31. In another example, mesenchymal lineage precursor or stem cells express STRO-1 and α2β1. In another example, mesenchymal lineage precursor or stem cells express STRO-1 and α4β1. In another example, mesenchymal lineage precursor or stem cells express STRO-1 and α5β1.

In another example, mesenchymal lineage precursor or stem cells according to the present disclosure express STRO-1 and an integrin having a (33 (CD61) chain polypeptide. In an example, the present disclosure encompasses a population of mesenchymal lineage precursor or stem cells enriched for STRO-1 and β3+cells. In this example, a population enriched for β3+cells comprises at least about 8% or 10% or 15% (33+cells. In another example, mesenchymal lineage precursor or stem cells express STRO-1 and αvβ3. In another example, mesenchymal lineage precursor or stem cells according to the present disclosure express STRO-1 and an integrin having a β5 (ITGB5) chain polypeptide. In an example, mesenchymal lineage precursor or stem cells express STRO-1 and αvβ5. In another example, mesenchymal lineage precursor or stem cells express STRO-1 and αvβ6.

Identifying and/or enriching for mesenchymal lineage precursor or stem cells expressing above referenced integrins may be achieved using various methods known in the art. In one example, fluorescent activated cell sorting (FACS) can be employed using commercially available antibodies (e.g. Thermofisher; Pharmingen; Abcam) to identify and select for cells expressing a desired integrin polypeptide chain or combination thereof.

In an example, mesenchymal lineage precursor or stem cells express STRO-1 and coxsackievirus and adenovirus receptor. In another example, mesenchymal lineage precursor or stem cells express STRO-1, coxsackievirus and adenovirus receptor and one or more of the above referenced integrin's.

In another example, mesenchymal lineage precursor or stem cells express STRO-1, coxsackievirus and adenovirus receptor, αvβ3 and αvβ5.

In an example, mesenchymal lineage precursor or stem cells are genetically modified to express one or more of the above referenced integrin's or coxsackievirus and adenovirus receptor on their cell surface.

In an example, mesenchymal lineage precursor or stem cells express STRO-1, a chimeric antigen receptor (CAR). For example, mesenchymal lineage precursor or stem cells express STRO-1, CAR, avf33 and avf35.

In an example, mesenchymal lineage precursor or stem cells expressing CAR can trigger a T cell mediated immune response. In another example, the CAR acts as a means of attaching mesenchymal lineage precursor or stem cells to cancer cells. In another example, the CAR acts as a means of triggering enhanced adhesion of mesenchymal lineage precursor or stem cells to cancer cells.

In an example, the CAR is comprised of an extracellular antigen binding domain, a transmembrane domain, and an intracellular domain. In an example, the antigen binding domain possesses affinity for one or more tumour antigens. Exemplary tumour antigens include HER2, CLPP, 707-AP, AFP, ART-4, BAGE, MAGE, GAGE, SAGE, b-catenin/m, bcr-abl, CAMEL, CAP-1, CEA, CASP-8, CDK/4, CDC-27, Cyp-B, DAM-8, DAM-10, ELV-M2, ETV6, G250, Gp100, HAGE, HER-2/neu, EPV-E6, LAGE, hTERT, survivin, iCE, MART-1, tyrosinase, MUC-1, MC1-R, TEL/AML, and WT-1.

Exemplary intracellular domains include CD3-zeta, CD28, 4-IBB, and the like, in some instances, the CAR can comprise any combination of CD3-zeta, CD28, 4-1 BB, TLR-4.

Exemplary transmembrane domains can be derived from (i.e. comprise at least the transmembrane region(s) of) the alpha, beta or zeta chain of the T-cell receptor, CD28, CD3 epsilon, CD45, CD4, CD5, CDS, CD9, CD 16, CD22, CD33, CD37, CD64, CD80, CD86, CD 134, CD137, 35 CD 154. In another example, the transmembrane domain can be synthetic, in which case it will comprise predominantly hydrophobic residues such as leucine and valine.

Mesenchymal lineage precursor or stem cells can be isolated from host tissues such as those referred to above and enriched for by immunoselection. For example, a bone marrow aspirate from a subject may be further treated with an antibody to STRO-1 or TNAP to enable selection of mesenchymal lineage precursor or stem cells. In one example, the mesenchymal lineage precursor or stem cells can be enriched for by using the STRO-1 antibody described in Simmons & Torok-Storb, 1991.

STRO-1+cells are cells found in bone marrow, blood, dental pulp cells, adipose tissue, skin, spleen, pancreas, brain, kidney, liver, heart, retina, brain, hair follicles, intestine, lung, lymph node, thymus, bone, ligament, tendon, skeletal muscle, dermis, and periosteum; and are capable of differentiating into germ lines such as mesoderm and/or endoderm and/or ectoderm. Thus, STRO-1+cells are capable of differentiating into a large number of cell types including, but not limited to, adipose, osseous, cartilaginous, elastic, muscular, and fibrous connective tissues. The specific lineage-commitment and differentiation pathway which these cells enter depends upon various influences from mechanical influences and/or endogenous bioactive factors, such as growth factors, cytokines, and/or local microenvironmental conditions established by host tissues.

The term “enriched” as used herein describes a population of cells in which the proportion of one particular cell type or the proportion of a number of particular cell types is increased when compared with an untreated population of the cells (e.g., cells in their native environment). In one example, a population enriched for STRO-1+cells comprises at least about 0.1% or 0.5% or 1% or 2% or 5% or 10% or 15% or 20% or 25% or 30% or 50% or 75% STRO-1+cells. In this regard, the term “population of cells enriched for STRO-1+cells” will be taken to provide explicit support for the term “population of cells comprising X% STRO-1+cells”, wherein X% is a percentage as recited herein. The STRO-1+cells can, in some examples, form clonogenic colonies, for example, CFU-F (fibroblasts) or a subset thereof (e.g., 50% or 60% or 70% or 70% or 90% or 95%) can have this activity.

In one example, the population of cells is enriched from a cell preparation comprising STRO-1+cells in a selectable form. In this regard, the term “selectable form” will be understood to mean that the cells express a marker (e.g., a cell surface marker) permitting selection of the STRO-1+cells. The marker can be STRO-1, but need not be. For example, as described and/or exemplified herein, cells (e.g., MPCs) expressing STRO-2 and/or STRO-3 (TNAP) and/or STRO-4 and/or VCAM-1 and/or CD146 and/or 3G5 also express STRO-1 (and can be STRO-lbright). Accordingly, an indication that cells are STRO-1+does not mean that the cells are selected by STRO-1 expression. In one example, the cells are selected based on at least STRO-3 expression, e.g., they are STRO-3+(TNAP+).

Reference to selection of a cell or population thereof does not necessarily require selection from a specific tissue source. As described herein, STRO-1+cells can be selected from or isolated from or enriched from a large variety of sources. That said, in some examples, these terms provide support for selection from any tissue comprising

STRO-1+cells or vascularized tissue or tissue comprising pericytes (e.g., STRO-1+or 3G5+pericytes) or any one or more of the tissues recited herein.

In one example, the mesenchymal lineage precursor or stem cells of the disclosure express one or more markers individually or collectively selected from the group consisting of TNAP+, VCAM-1+, THY-1+, STRO-2+, STRO-4+(HSP-90(3), CD45+, CD146+, 3G5+.

By “individually” is meant that the disclosure encompasses the recited markers or groups of markers separately, and that, notwithstanding that individual markers or groups of markers may not be separately listed herein, the accompanying claims may define such marker or groups of markers separately and divisibly from each other.

By “collectively” is meant that the disclosure encompasses any number or combination of the recited markers or groups of markers, and that, notwithstanding that such numbers or combinations of markers or groups of markers may not be specifically listed herein, the accompanying claims may define such combinations or sub-combinations separately and divisibly from any other combination of markers or groups of markers.

A cell that is referred to as being “positive” for a given marker may express either a low (lo or dim or dull), intermediate (median) or a high (bright, bri) level of that marker depending on the degree to which the marker is present on the cell surface, where the terms relate to intensity of fluorescence or other marker used in the sorting process of the cells or flow cytometric analysis of the cells. The distinction of low (lo or dim or dull), intermediate (median), or high (bright, bri) will be understood in the context of the marker used on a particular cell population being sorted or analysed. A cell that is referred to as being “negative” for a given marker is not necessarily completely absent from that cell. This term means that the marker is expressed at a relatively very low level by that cell, and that it generates a very low signal when detectably labelled or is undetectable above background levels, for example, levels detected using an isotype control antibody.

The term “bright” or bri as used herein, refers to a marker on a cell surface that generates a relatively high signal when detectably labelled. Whilst not wishing to be limited by theory, it is proposed that “bright” cells express more of the target marker protein (for example, the antigen recognized by a STRO-1 antibody) than other cells in the sample. For instance, STRO-lbri cells produce a greater fluorescent signal, when labelled with a FITC-conjugated STRO-1 antibody as determined by fluorescence activated cell sorting (FACS) analysis, than non-bright cells (STRO-11o/dim/dull/intermediate/median). In one example, the mesenchymal lineage precursor or stem cells are isolated from bone marrow and enriched for by selection of STRO-1+ cells. In this example, “bright” cells constitute at least about 0.1% of the most brightly labelled bone marrow mononuclear cells contained in the starting sample. In other examples, “bright” cells constitute at least about 0.1%, at least about 0.5%, at least about 1%, at least about 1.5%, or at least about 2%, of the most brightly labelled bone marrow mononuclear cells contained in the starting sample. In an example, STRO-lbright cells have 2 log magnitude higher expression of STRO-1 surface expression relative to “background”, namely cells that are STRO-1-. By comparison, STRO-11o/dim/dull and/or STRO-lintermediate/median cells have less than 2 log magnitude higher expression of STRO-1 surface expression, typically about 1 log or less than “background”.

In one example, the STRO-1+cells are STRO-lbright. In one example, the STRO-lbright cells are preferentially enriched relative to STRO-11o/dim/dull or STRO-lintermediate/median cells.

In one example, the STRO-lbright cells are additionally one or more of TNAP+, VCAM-1+, THY-1+, STRO-2+, STRO-4+(HSP-90(3) and/or CD146+. For example, the cells are selected for one or more of the foregoing markers and/or shown to express one or more of the foregoing markers. In this regard, a cell shown to express a marker need not be specifically tested, rather previously enriched or isolated cells can be tested and subsequently used, isolated or enriched cells can be reasonably assumed to also express the same marker.

In one example, the STRO-lbright cells are perivascular mesenchymal lineage precursor or stem cells as defined in WO 2004/85630, characterized by the presence of the perivascular marker 3G5.

As used herein the term “TNAP” is intended to encompass all isoforms of tissue non-specific alkaline phosphatase. For example, the term encompasses the liver isoform (LAP), the bone isoform (BAP) and the kidney isoform (KAP). In one example, the TNAP is BAP. In one example, TNAP refers to a molecule which can bind the STRO-3 antibody produced by the hybridoma cell line deposited with ATCC on 19 December 2005 under the provisions of the Budapest Treaty under deposit accession number PTA-7282.

Furthermore, in one example, the STRO-1+cells are capable of giving rise to clonogenic CFU-F.

In one example, a significant proportion of the STRO-1+cells are capable of differentiation into at least two different germ lines. Non-limiting examples of the lineages to which the cells may be committed include bone precursor cells; hepatocyte progenitors, which are multipotent for bile duct epithelial cells and hepatocytes; neural restricted cells, which can generate glial cell precursors that progress to oligodendrocytes and astrocytes; neuronal precursors that progress to neurons; precursors for cardiac muscle and cardiomyocytes, glucose-responsive insulin secreting pancreatic beta cell lines. Other lineages include, but are not limited to, odontoblasts, dentin-producing cells and chondrocytes, and precursor cells of the following: retinal pigment epithelial cells, fibroblasts, skin cells such as keratinocytes, dendritic cells, hair follicle cells, renal duct epithelial cells, smooth and skeletal muscle cells, testicular progenitors, vascular endothelial cells, tendon, ligament, cartilage, adipocyte, fibroblast, marrow stroma, cardiac muscle, smooth muscle, skeletal muscle, pericyte, vascular, epithelial, glial, neuronal, astrocyte and oligodendrocyte cells.

In one example, the mesenchymal lineage precursor or stem cells are MSCs. The

MSCs may be a homogeneous composition or may be a mixed cell population enriched in MSCs. Homogeneous MSC compositions may be obtained by culturing adherent bone marrow or periosteal cells, and the MSCs may be identified by specific cell surface markers which are identified with unique monoclonal antibodies. A method for obtaining a cell population enriched in MSCs is described, for example, in US patent 5486359. MSC prepared by conventional plastic adherence isolation relies on the non-specific plastic adherent properties of CFU-F. Mesenchymal lineage precursor or stem cells isolated from bone marrow by immunoselection based on STRO-1 specifically isolates clonogenic mesenchymal precursors from bone marrow populations in the absence of other plastic adherent bone marrow populations. Alternative sources for MSCs include, but are not limited to, blood, skin, cord blood, muscle, fat, bone, and perichondrium. In an example, the MSCs are allogeneic. In an example, the MSCs are cryopreserved. In an example, the MSCs are culture expanded and cryopreserved.

In one example, the mesenchymal lineage precursor or stem cells are derived from pluripotent cells such as induced pluripotent stem cells (iPS cells). In one embodiment the pluripotent cells are human pluripotent cells. Suitable processes for generation of mesenchymal lineage precursor or stem cells from pluripotent cells are described, for example, in US 7,615,374 and US 2014273211, Barberi et al; Plos medicine, Vol 2(6):0554-0559 (2005), and Vodyanik et al. Cell Stem cell, Vol 7:718-728 (2010).

In another example, the mesenchymal lineage precursor or stem cells are immortalised. Exemplary processes for generation of immortalised mesenchymal lineage precursor or stem cells are described, for example, in Obinata M., Cell, Vol 2:235-244 (1997), US 9,453,203, Akimov et al. Stem Cells, Vol 23:1423-1433 and Kabara et al. Laboratory Investigation, Vol 94: 1340-1354 (2014).

In a preferred embodiment of the present disclosure, the mesenchymal lineage precursor or stem cells are obtained from a master cell bank derived from mesenchymal lineage precursor or stem cells enriched from the bone marrow of healthy volunteers. The use of mesenchymal lineage precursor or stem cells derived from such a source is particularly advantageous for subjects who do not have an appropriate family member available who can serve as the mesenchymal lineage precursor or stem cell donor, or are in need of immediate treatment and are at high risk of relapse, disease-related decline or death, during the time it takes to generate mesenchymal lineage precursor or stem cells.

In another example, mesenchymal lineage precursor cells express Cx43. In another example, mesenchymal lineage precursor cells express Cx40. In another example, mesenchymal lineage precursor cells express Cx43 and Cx40. In another example, mesenchymal lineage precursor cells express Cx45, Cx32 and/or Cx37. In an example, mesenchymal lineage precursor cells are not modified to express a particular connexin.

Isolated or enriched mesenchymal lineage precursor cells can be expanded in vitro by culture. Isolated or enriched mesenchymal lineage precursor cells can be cryopreserved, thawed and subsequently expanded in vitro by culture.

In one example, isolated or enriched mesenchymal lineage precursor cells are seeded at 50,000 viable cells/cm² in culture medium (serum free or serum-supplemented), for example, alpha minimum essential media (aMEM) supplemented with 5% fetal bovine serum (FBS) and glutamine, and allowed to adhere to the culture vessel overnight at 37° C., 20% 0². The culture medium is subsequently replaced and/or altered as required and the cells cultured for a further 68 to 72 hours at 37° C., 5% 0².

As will be appreciated by those of skill in the art, cultured mesenchymal lineage precursor cells are phenotypically different to cells in vivo. For example, in one embodiment they express one or more of the following markers, CD44, NG2, DC146 and CD140b. Cultured mesenchymal lineage precursor cells are also biologically different to cells in vivo, having a higher rate of proliferation compared to the largely non-cycling (quiescent) cells in vivo.

In an example, mesenchymal lineage precursor or stem cells are obtained from a single donor, or multiple donors where the donor samples or mesenchymal lineage precursor or stem cells are subsequently pooled and then culture expanded.

Mesenchymal lineage precursor or stem cells encompassed by the present disclosure may also be cryopreserved prior to administration to a subject. In an example, mesenchymal lineage precursor or stem cells are culture expanded and cryopreserved prior to administration to a subject.

In an example, the present disclosure encompasses mesenchymal lineage precursor or stem cells as well as progeny thereof, soluble factors derived therefrom, and/or extracellular vesicles isolated therefrom. In another example, the present disclosure encompasses mesenchymal lineage precursor or stem cells as well as extracellular vesicles isolated therefrom. For example, it is possible to culture expand mesenchymal precursor lineage or stem cells of the disclosure for a period of time and under conditions suitable for secretion of extracellular vesicles into the cell culture medium. Secreted extracellular vesicles can subsequently be obtained from the culture medium for use in therapy.

The term “extracellular vesicles” as used herein, refers to lipid particles naturally released from cells and ranging in size from about 30 nm to as a large as 10 microns, although typically they are less than 200 nm in size. They can contain proteins, nucleic acids, lipids, metabolites, or organelles from the releasing cells (e.g., mesenchymal stem cells; STRO-1⁺cells).

The term “exosomes” as used herein, refers to a type of extracellular vesicle generally ranging in size from about 30 nm to about 150 nm and originating in the endosomal compartment of mammalian cells from which they are trafficked to the cell membrane and released. They may contain nucleic acids (e.g., RNA; microRNAs), proteins, lipids, and metabolites and function in intercellular communication by being secreted from one cell and taken up by other cells to deliver their cargo.

### Culture Expansion of the Cells

In an example, mesenchymal lineage precursor or stem cells are culture expanded. “Culture expanded” mesenchymal lineage precursor or stem cells media are distinguished from freshly isolated cells in that they have been cultured in cell culture medium and passaged (i.e. sub-cultured). In an example, culture expanded mesenchymal lineage precursor or stem cells are culture expanded for about 4 — 10 passages. In an example, mesenchymal lineage precursor or stem cells are culture expanded for at least 5, at least 6, at least 7, at least 8, at least 9, at least 10 passages. For example, mesenchymal lineage precursor or stem cells can be culture expanded for at least 5 passages. In an example, mesenchymal lineage precursor or stem cells can be culture expanded for at least 5 — 10 passages. In an example, mesenchymal lineage precursor or stem cells can be culture expanded for at least 5 — 8 passages. In an example, mesenchymal lineage precursor or stem cells can be culture expanded for at least 5 — 7 passages. In an example, mesenchymal lineage precursor or stem cells can be culture expanded for more than 10 passages. In another example, mesenchymal lineage precursor or stem cells can be culture expanded for more than 7 passages. In these examples, stem cells may be culture expanded before being cryopreserved to provide an intermediate cryopreserved MLPSC population. In an example, compositions of the present disclosure are produced by culturing cells from an intermediate cryopreserved MLPSC population or, put another way, a cryopreserved intermediate.

In an example, compositions of the disclosure comprise mesenchymal lineage precursor or stem cells that are culture expanded from a cryopreserved intermediate. In an example, the cells culture expanded from a cryopreserved intermediate are culture expanded for at least 5, at least 6, at least 7, at least 8, at least 9, at least 10 passages. For example, mesenchymal lineage precursor or stem cells can be culture expanded for at least 5 passages. In an example, mesenchymal lineage precursor or stem cells can be culture expanded for at least 5 — 10 passages. In an example, mesenchymal lineage precursor or stem cells can be culture expanded for at least 5 — 8 passages. In an example, mesenchymal lineage precursor or stem cells can be culture expanded for at least 5 — 7 passages. In an example, mesenchymal lineage precursor or stem cells can be culture expanded for more than 10 passages. In another example, mesenchymal lineage precursor or stem cells can be culture expanded for more than 7 passages.

In an example, mesenchymal lineage precursor or stem cells culture expanded from a cryopreserved intermediate can be culture expanded in medium free of animal proteins. In an example, mesenchymal lineage precursor or stem cells culture expanded from a cryopreserved intermediate can be culture expanded in xeno-free medium. In an example, mesenchymal lineage precursor or stem cells culture expanded from a cryopreserved intermediate can be culture expanded in medium that is fetal bovine serum free.

In an embodiment, mesenchymal lineage precursor or stem cells can be obtained from a single donor, or multiple donors where the donor samples or mesenchymal lineage precursor or stem cells are subsequently pooled and then culture expanded. In an example, the culture expansion process comprises:

i. expanding by passage expansion the number of viable cells to provide a preparation of at least about 1 billion of the viable cells, wherein the passage expansion comprises establishing a primary culture of isolated mesenchymal lineage precursor or stem cells and then serially establishing a first non-primary (P1) culture of isolated mesenchymal lineage precursor or stem cells from the previous culture;

ii. expanding by passage expansion the P1 culture of isolated mesenchymal lineage precursor or stem cells to a second non-primary (P2) culture of mesenchymal lineage precursor or stem cells; and,

iii. preparing and cryopreserving an in-process intermediate mesenchymal lineage precursor or stem cells preparation obtained from the P2 culture of mesenchymal lineage precursor or stem cells; and,

iv. thawing the cryopreserved in-process intermediate mesenchymal lineage precursor or stem cells preparation and expanding by passage expansion the in-process intermediate mesenchymal lineage precursor or stem cells preparation.

In an example, the expanded mesenchymal lineage precursor or stem cell preparation has an antigen profile and an activity profile comprising:

i. less than about 0.75% CD45+cells;

ii. at least about 95% CD105+cells;

iii. at least about 95% CD166+cells.

In an example, the expanded mesenchymal lineage precursor or stem cell preparation is capable of inhibiting IL2Ra expression by CD3/CD28-activated PBMCs by at least about 30% relative to a control.

In an example, culture expanded mesenchymal lineage precursor or stem cells are culture expanded for about 4 — 10 passages, wherein the mesenchymal lineage precursor or stem cells have been cryopreserved after at least 2 or 3 passages before being further culture expanded. In an example, mesenchymal lineage precursor or stem cells are culture expanded for at least 1, at least 2, at least 3, at least 4, at least 5 passages, cryopreserved and then further culture expanded for at least 1, at least 2, at least 3, at least 4, at least 5 passages before being cultured according to the methods of the disclosure.

The process of mesenchymal lineage precursor or stem cell isolation and ex vivo expansion can be performed using any equipment and cell handing methods known in the art. Various culture expansion embodiments of the present disclosure employ steps that require manipulation of cells, for example, steps of seeding, feeding, dissociating an adherent culture, or washing. Any step of manipulating cells has the potential to insult the cells. Although mesenchymal lineage precursor or stem cells can generally withstand a certain amount of insult during preparation, cells are preferably manipulated by handling procedures and/or equipment that adequately performs the given step(s) while minimizing insult to the cells.

In an example, mesenchymal lineage precursor or stem cells are washed in an apparatus that includes a cell source bag, a wash solution bag, a recirculation wash bag, a spinning membrane filter having inlet and outlet ports, a filtrate bag, a mixing zone, an end product bag for the washed cells, and appropriate tubing, for example, as described in U.S Pat. No. 6,251,295, which is hereby incorporated by reference.

In an example, a mesenchymal lineage precursor or stem cell composition cultured according to the present disclosure is 95% homogeneous with respect to being CD105 positive and CD166 positive and being CD45 negative. In an example, this homogeneity persists through ex vivo expansion; i.e. though multiple population doublings.

In an example, mesenchymal lineage precursor or stem cells of the disclosure are culture expanded in 3D culture. For example, mesenchymal lineage precursor or stem cells of the disclosure can be culture expanded in a bioreactor. In an example, mesenchymal lineage precursor or stem cells of the disclosure are initially culture expanded in 2D culture prior to being further expanded in 3D culture. In an example, mesenchymal lineage precursor or stem cells of the disclosure are culture expanded from a master cell bank. In an example, mesenchymal lineage precursor or stem cells of the disclosure are culture expanded from a master cell bank in 2D culture before seeding in 3D culture. In an example, mesenchymal lineage precursor or stem cells of the disclosure are culture expanded from a master cell bank in 2D culture for at least 3 days before seeding in 3D culture in a bioreactor. In an example, mesenchymal lineage precursor or stem cells of the disclosure are culture expanded from a master cell bank in 2D culture for at least 4 days before seeding in 3D culture in a bioreactor. In an example, mesenchymal lineage precursor or stem cells of the disclosure are culture expanded from a master cell bank in 2D culture for between 3 and 5 days before seeding in 3D culture in a bioreactor. In these examples, 2D culture can be performed in a cell factory. Various cell factory products are available commercially (e.g. Thermofisher, Sigma).

### Ang1 and VEGF Levels

In another aspect, mesenchymal lineage precursor or stem cells according to the present disclosure express Angl:VEGF at a ratio of at least about 2:1. However, in other examples, mesenchymal lineage precursor or stem cells express Angl:VEGF at a ratio of at least about 10:1, 15:1, 20:1, 21:1, 22:1, 23:1, 24:1, 25:1, 26:1, 27:1, 28:1, 29:1, 30:1, 31:1, 32:1, 33:1, 34:1, 35:1, 50:1.

The amount of cellular Ang1 and/or VEGF that is expressed in a composition or culture of mesenchymal lineage precursor or stem cells may be determined by methods known to those skilled in the art. Such methods include, but are not limited to, quantitative assays such as quantitative ELISA assays, for example. In this example, a cell lysate from a culture of mesenchymal lineage precursor or stem cells is added to a well of an ELISA plate. The well may be coated with a primary antibody, either a monoclonal or a polyclonal antibody(ies), against the Ang1 or VEGF. The well then is washed, and then contacted with a secondary antibody, either a monoclonal or a polyclonal antibody(ies) , against the primary antibody. The secondary antibody is conjugated to an appropriate enzyme, such as horseradish peroxidase, for example. The well then may be incubated, and then is washed after the incubation period. The wells then are contacted with an appropriate substrate for the enzyme conjugated to the secondary antibody, such as one or more chromogens. Chromogens which may be employed include, but are not limited to, hydrogen peroxide and tetramethylbenzidine. After the substrate(s) is (are) added, the well is incubated for an appropriate period of time. Upon completion of the incubation, a “stop” solution is added to the well in order to stop the reaction of the enzyme with the substrate(s). The optical density (OD) of the sample is then measured. The optical density of the sample is correlated to the optical densities of samples containing known amounts of Ang1 or VEGF in order to determine the amount of Ang1 or VEGF expressed by the culture of stem cells being tested.

Methods for determining the Angl:VEGF expression ratio will also be apparent to one of skill in the art. For example Ang1 and VEGF expression levels can be quantitated via quantitative ELISA as discussed above. After quantifying the levels of Ang1 and VEGF, a ratio based on the quantitated levels of Ang1 and VEGF could be represented as: (level of Ang1/level of VEGF) =Angl:VEGF ratio.

In an example, the mesenchymal lineage precursor or stem cells of the present disclosure are not genetically modified to express Ang1 and/or VEGF at an above exemplified level or ratio. Cells that are not genetically modified to express Ang1 and/or VEGF have not been modified by transfection with a nucleic acid expressing or encoding Ang1 and/or VEGF. For the avoidance of doubt, in the context of the present disclosure a mesenchymal lineage precursor or stem cell transfected with a nucleic acid encoding Ang1 and/or VEGF would be considered genetically modified. In the context of the present disclosure cells not genetically modified to express Ang1 and/or VEGF naturally express Ang1 and/or VEGF to some extent without transfection with a nucleic acid encoding Ang1 and/or VEGF1.

### Recombinant Virus

In an embodiment cells defined herein are modified to introduce a recombinant virus. The term “recombinant virus” is used in the context of the present disclosure to refer to viruses that express a transgene of interest in a cell (or population thereof) defined herein. In an example, a recombinant virus expresses a transgene that increases migration of mesenchymal precursor or stem cells to cancer cells. In an example, the recombinant virus comprises a herpes simplex virus backbone. In an example, the recombinant virus is a herpes simplex virus.

The term “oncolytic virus” is used in the context of the present disclosure to refer to viruses that are able to infect and reduce growth of cancer cells. For example, oncolytic viruses can inhibit cell proliferation. In another example, oncolytic viruses can kill cancer cells. In an example, the oncolytic virus preferentially infects and inhibits growth of cancer cells compared with corresponding normal cells. In another example, the oncolytic virus preferentially replicates in and inhibits growth of cancer cells compared with corresponding normal cells.

In an example, the oncolytic virus is able to naturally infect and reduce growth of cancer cells. Examples of such viruses include Newcastle disease virus, vesicular stomatitis, myxoma, reovirus, sindbis, measles and coxsackievirus. Oncolytic viruses able to naturally infect and reduce growth of cancer cells generally target cancer cells by exploiting the cellular aberrations that occur in these cells. For example, oncolytic viruses may exploit surface attachment receptors, activated oncogenes such as Ras, Akt, p53 and/or interferon (IFN) pathway defects.

In another example, oncolytic viruses encompassed by the present disclosure are engineered to infect and reduce growth of cancer cells. Exemplary viruses suitable for such engineering include oncolytic DNA viruses, such as adenovirus, herpes simplex virus (HSV) and Vaccinia virus; and oncolytic RNA viruses such as Lentivirus, Reovirus, Coxsackievirus, Seneca Valley Virus, Poliovirus, Measles virus, Newcastle disease virus, Vesicular stomatitis virus (VSV) and parvovirus such as rodent protoparvoviruses H-1PV. In an example, the oncolytic virus comprises a backbone of an above referenced virus. For example, the oncolytic virus can comprise a HSV backbone. In an example, the oncolytic virus is a HSV.

In an example, tumour specificity of an oncolytic virus can be engineered to mutate or delete gene(s) required for survival of the virus in normal cells but expendable in cancer cells. For the avoidance of doubt, oncolytic viruses with mutated or deleted genes are able to survive in mesenchymal lineage precursor or stem cells for a sufficient duration to allow transfer to a cancer cell. For example, the oncolytic virus can be engineered by mutating or deleting a gene that encodes thymidine kinase, an enzyme needed for nucleic acid metabolism. In this example, viruses are dependent on cellular thymidine kinase expression, which is high in proliferating cancer cells but repressed in normal cells. In another example, the oncolytic virus is engineered to comprise a capsid protein that binds a tumour specific cell surface molecule. In an example, the capsid protein is a fibre, a penton or hexon protein. In another example, the oncolytic virus is engineered to comprise a tumour specific cell surface molecule for transductionally targeting a cancer cell. Exemplary tumour specific cell surface molecules can include an integrin, an EGF receptor family member, a proteoglycan, a disialoganglioside, B7-H3, CA-125, EpCAM, ICAM-1, DAF, A21, integrin-a2(31, vascular endothelial growth factor receptor 1 , vascular endothelial growth factor receptor 2, CEA, a tumour associated glycoprotein, CD19, CD20, CD22, CD30, CD33, CD40, CD44, CD52, CD74, CD152, CD155, MUC1, a tumour necrosis factor receptor, an insulin-like growth factor receptor, folate receptor a, transmembrane glycoprotein NMB, a C-C chemokine receptor, PSMA, RON-receptor, and cytotoxic T-lymphocyte antigen 4.

In another example, the oncolytic virus is engineered to increase capacity of an infected mesenchymal lineage precursor or stem cell to deliver viral payload to cancer cells. For example, the oncolytic virus can be engineered to express a viral fusogenic membrane glycoprotein to mediate induction of mesenchymal precursor lineage or stem cell fusion to cancer cells. Examples, of viral fusogenic membrane glycoproteins include gibbon-ape leukaemia virus (GLAV) envelope glycoprotein, measles virus protein F (MV-F) and measles virus protein H (MV-H).

In an example, the viral fusogenic membrane glycoprotein is under control of a late promoter such as adenovirus major late promoter. In an example, the viral fusogenic membrane glycoprotein is under control of a strict late promoter such as UL38p (WO 2003/082200) which is only active after the start of viral DNA replication. Examples of such promoters and engineered viruses are disclosed in Fu et al. (2003) Molecular Therapy, 7:748-54 and Guedan et al. (2012) Gene Therapy, 19:1048-1057.

In an example, the oncolytic virus is replication-competent. In an example, oncolytic viruses selectively replicate in cancer cells when compared with corresponding normal cells and/or mesenchymal lineage precursor or stem cells. In an example, tumour specificity of oncolytic virus can be engineered to restrict virus replication by its dependence on transcriptional activities that are constitutively activated in cancer cells (i.e. conditional replication). In an example, the oncolytic virus is a conditionally replicative lentivirus. In another example, the oncolytic virus is a conditionally replicative adenovirus, reovirus, measles, herpes simplex virus, Newcatle disease virus or vaccinia.

In an example, conditional replication is achieved by the insertion of a tumour-specific promoter driving the expression of a critical gene(s). Such promoters can be identified based on differences in gene expression between tumour, corresponding surrounding tissue and/or mesenchymal lineage precursor or stem cells. For example, one way of identifying an appropriate tumour specific promoter is to compare gene expression levels between tumour, corresponding normal tissue and mesenchymal lineage precursor or stem cells to identify those genes that are expressed at high levels in tumour and low levels in the corresponding healthy tissue and/or mesenchymal lineage precursor or stem cells. Tumour specific promoters may be native or composite. Exemplary native promoters include AFP, CCKAR, CEA, erbB2, Cerb2, COX2, CXCR4, E2F1, HE4, LP, MUC1, PSA, Survivin, TRP1, STAT3, hTERT and Tyr. Exemplary composite promoters include AFP/hAFP, SV40/AFP, CEA/CEA, PSA/PSA, SV40/Tyr and Tyr/Tyr. One of skill in the art will appreciate that the appropriate tumour specific promoter will in some instances be dictated by the target tumour. For example, a cerb2 promoter may be appropriate for breast and pancreatic cancers while a PSA promoter may be appropriate for prostate cancers.

In another example, tumour specific promoters can be identified based on differences in promoter activity in cancer cells compared with corresponding normal cells and/or mesenchymal lineage precursor or stem cells. For example, one way of identifying an appropriate tumour specific promoter is to compare promoter activity between cancer cells, corresponding normal cells and/or mesenchymal lineage precursor or stem cells to identify those promoters with high activity in cancer cells and low activity in corresponding normal cells and/or mesenchymal lineage precursor or stem cells. In an example, the tumour specific promoter may be a late or strict-late viral promoter. The terms “late” and “strict-late” are used to refer to promoters whose activity depends on the initiation of viral DNA replication. Thus, late and strict-late promoters are suitable for inclusion in oncolytic viruses that can replicate in cancer cells but have limited ability to replicate in non-dividing normal cells. Exemplary late or strict late promoters include major late promoter (MLP) and UL38p.

In an example, the oncolytic virus is a herpes simplex virus or adenovirus comprising a late or strict late promoter. For example, the oncolytic virus is a herpes simplex virus comprising an UL38p promoter. In another example, the oncolytic virus is an adenovirus comprising a MLP.

In another example, tumour specificity of oncolytic virus can be engineered to exploit a tumour specific tropism. In another example, the oncolytic virus is sensitive to an oligonucleotide or binding protein expressed in normal cells and/or mesenchymal lineage precursor or stem cells that is expressed at low levels or is absent in cancer cells. For example, the oncolytic virus can be engineered to insert a nucleotide sequence that is complimentary to an oligonucleotide that is expressed by mesenchymal lineage precursor or stem cells and/or normal cells and not expressed by cancer cells. For example, the oncolytic virus can be sensitive to an inhibitory oligonucleotide such as a miRNA. Exemplary miRNAs expressed at low levels in some cancer cells and high levels in corresponding normal cells may include let-7a-5p, miR-122-5p, miR-125b-5p, miR-141-3p, miR-143-3p, miR-15a-5p, miR-16-5p, miR-181a-5p, miR-181b-5p, miR-192-5p, miR-195-5p, miR-200b-3p, miR-200c-3p, miR-211-5p, miR-215-5p, miR-22-3p, miR-29a-3p, miR-29b-3p, miR-29c-3p, miR-30a-5p, miR-30c-5p, miR-34a-5p, miR-34c-5p, miR-424-5p, miR-497-5p, miR-7-5p, miR-101-3p, miR-124-3p, miR-126-3p, miR-137, miR-138-5p, miR-140-5p, miR-152-3p, miR-185-5p, miR-214-3p, miR-25-3p, miR-26a-5p, miR-26b-5p, miR-3′72-3p, miR-517a-3p, miR-520c-3p, miR-128-3p, miR-145-5p, miR-200a-3p, miR-502-5p, let-7d-5p, let-7e-5p, let-7f-5p, miR-155-5p, miR-98-5p, let-7b-5p, miR-1, miR-100-5p, miR-125a-5p, miR-133a-3p, miR-133b, miR-146a-5p, miR-150-5p, miR-193a-3p, miR-193b-3p, miR-196b-5p, miR-206, miR-218-5p, miR-223-3p, miR-23b-3p, miR-24-3p, miR-34b-3p, miR-449a, miR-542-5p, miR-99a-5p, let-7c-5p, let-7g-5p, let-7i-5p, miR-142-3p, miR-216b-5p, miR-622, miR-96-5p, miR-1291, miR-3′70-3p, miR-296-5p, miR-335-5p, miR-483-3p, miR-483-5p, miR-486-5p.

In another example, the virus can be engineered to express a gene(s) in infected cancer cells. For example, the virus can be engineered to expresses a gene(s) such as PTEN. In an example, the virus expresses PTEN-alpha (PTENα). In an example, the virus comprises a nucleic acid sequence as shown in SEQ ID NO: 1 or a variant thereof that is translated into a functional PTEN protein (e.g. SEQ ID NO: 2). In an example, the virus expresses a transgene that is expressed and translated into a protein having an amino acid as shown in SEQ ID NO: 2. In these examples, the oncolytic virus can comprise a HSV backbone. In an example, the virus is a HSV. In an example, the HSV is as described in Russell et al. (2018) Nat Comm., 9:5006. In these examples, the virus can increase levels of protein having an amino acid as shown in SEQ ID NO: 2 in infected cancer cells.

In an example, the gene(s) enhance the immune response against an infected tumour cell. For example, the gene(s) may be GM-CSF, FLT3L, CCL3, CCL5, IL2, IL4, IL6, IL12, IL15, IL 18, IFNA1, IFNB1, IFNG, CD80, 4-1BBL, CD4OL, a heatshock protein (HSP) or a combination thereof

Various viruses may be engineered as outlined in the above referenced examples. In an example, the oncolytic virus is a modified HSV, Lentivirus, Baculovirus, Retrovirus, Adenovirus (AdV), Adeno-associated virus (AAV) or a recombinant form such as recombinant adeno-associated virus (rAAV) and derivatives thereof such as self-complementary AAV (scAAV) and non-integrating AV. For example, the oncolytic virus can be a modified HSV. For example, the oncolytic virus can be a modified lentivirus. Other exemplary viruses include vaccina virus, vesicular stomatitis virus (VSV), measles virus and maraba virus.

In other examples, the oncolytic virus may be one of various AV or AAV serotypes. In an example, the oncolytic virus is serotype 1. In another example, the oncolytic virus is serotype 2. In other examples, the oncolytic virus is serotype 3, 4, 7, 8, 9, 10, 11, 12 or 13. In another example, the oncolytic virus is serotype 5. In another example, the oncolytic virus is serotype 6.

Exemplary oncolytic viruses that may be introduced into mesenchymal lineage precursor or stem cells according to the present disclosure include T-Vec (HSV-1; Amgen), JX-594 (Vaccina; Sillaj en), JX-594 (AdV; Cold Genesys), Reolysin (Reovirus; Oncolytics Biotech). Other examples of oncolytic viruses are disclosed in WO 2003/080083, WO 2005/086922, WO 2007/088229, WO 2008/110579, WO 2010/108931, WO 2010/128182, WO 2013/112942, WO 2013/116778, WO 2014/204814, WO 2015/077624 and WO 2015/166082, WO 2015/089280.

In an example, the oncolytic virus is replication-defective. For example, replication genes can be mutated, deleted or replaced with an expression cassette with a tumour specific promoter. In an example, E1/E3 genes are mutated, deleted or replaced. In another example, E1A/E1B genes are mutated, deleted or replaced. For example, in the context of AV, E1/E3 genes can be mutated, deleted or replaced. In the context of AAV, E1A and E1B genes can be mutated, deleted or replaced. Various examples of suitable tumour specific promoters are discussed above.

In other examples, the oncolytic virus can comprise a mutated El, E3, E1A or E1B gene. For example, the E1A gene can be mutated in the region coding for the retinoblastoma protein (RB) binding site. In another example, the E3 gene can be mutated in the region coding for the endoplasmic reticulum retention domain. In another example, the oncolytic virus can comprise a mutation in the gamma-34.5 gene and/or the alpha-47 gene.

In an example, the oncolytic virus is replication-defective in a mesenchymal lineage precursor or stem cell and replication-competent in a tumour cell. An example, of switching a replication-defective virus into a replication-competent virus is described in Nakashima et al. (2014) Journal of Virology, Vol 88:345-353. Other exemplary viruses of this type include RGD mutants such as those described in Shen et al. (2016) PlosOne 11:e0147173, viruses comprising delta 24 mutation in El that enables replication in pRb or p53 inactive cancer cells and/or regulated expression of El under control of tumour cell specific promoters such as a-chemokine SDF-1 receptor (CXCR4), survivin, cyclooxygenase-2 (COX-2), and midkine.

In an example, viruses disclosed herein comprise a polynucleotide operatively linked to a tumour specific promotor. For example, the virus can comprise a polynucleotide encoding PTENα operatively linked to a tumour specific promotor.

In another example, viruses disclosed herein comprise a polynucleotide operatively linked to a constitutive promotor. For example, the virus can comprise a polynucleotide encoding PTENα operatively linked to a constitutive promotor.

### Modification

Mesenchymal lineage precursor or stem cells of the present disclosure can be modified to enhance cancer cell killing and/or migration towards cancer cells. In an example, such modification comprises increasing expression of PTENα. Various examples of modifications which increase gene expression are known in the art. For example, cells disclosed herein can be modified using a vector expressing a transgene such as a viral vector. Accordingly, in an example, mesenchymal lineage precursor or stem cells of the present disclosure can be modified to introduce a recombinant virus expressing a transgene. For example, mesenchymal lineage precursor or stem cells of the present disclosure can be modified to introduce a recombinant virus such as a virus comprising a HSV backbone and expressing a polynucleotide encoding PTENα. In an example, mesenchymal lineage precursor or stem cells are considered “modified” when a virus has been transferred into the cell by any suitable means of artificial manipulation, or where the cell is a progeny of an originally altered cell that carries the virus. In an example, cells transfected with a “naked” nucleic acid molecule encoding a transgene are not considered “modified”. For example, cells transfected with a “naked” mRNA molecule encoding a transgene are not considered “modified”.

In other examples, cell populations modified to introduce a recombinant virus can also be modified to express binding proteins such as antibodies or fragments thereof on the cell surface. For example, cell populations can also be modified to express an anti-epidermal growth factor receptor (EGFR; ErbB1) binding protein.

Enhanced migration of modified cells disclosed herein can be assessed using various migration assays known in the art such as transwell cell migration and invasion assay (see e.g. Justus et al. 2014 J Vis Exp., 88:51046 for summary; commercially available from suppliers such as Sigma and Merck; live cell analysis systems are also commercially available and appropriate for tracking migration in real time). Enhanced killing of cancer cells disclosed herein can also be assessed using various assays known in the art such as the cell viability/cytotoxicity assays exemplified below (e.g. assessment of cytosolic activity (aqua live/dead dye) by flow cytometry; see also e.g. Riss et al. (2013) Assay Guidance Manual., Last Update July 2016; commercially available kits from suppliers such as Promega; live cell analysis systems are also commercially available and appropriate for tracking cell viability in real time). Similarly increased gene expression can be quantified using various methods such as routine amplification based detection techniques including, for example, Real-Time polymerase chain reaction. In some cases, increased protein expression corresponding to increased gene expression can also be quantified using routine methods such as Western Blot.

Mesenchymal lineage precursor or stem cells can be modified using various methods known in the art. In an example, mesenchymal lineage precursor or stem cells are contacted with a virus in vitro. For example, virus can be added to mesenchymal lineage precursor or stem cell culture medium. In another example, mesenchymal lineage precursor or stem cells are centrifuged with virus.

Efficiencies of infection are rarely 100%, and it is usually desirable to enrich the population for cells that have been successfully modified. In an example, modified cells can be enriched by taking advantage of a functional feature of the new genotype. One exemplary method of enriching modified cells is positive selection using resistance to a drug such as neomycin or colorimetric selection based on expression of lacZ. The present inventors have found HSV expressing PTEN-alpha transgene has a high rate of infectivity in mesenchymal lineage precursor or stem cells. For example, HSV according to the present disclosure has at least 15% infectivity. In another example, HSV according to the present disclosure has at least 20% infectivity. In another example, HSV according to the present disclosure has at least 25% infectivity. In another example, HSV according to the present disclosure has at least 30% infectivity. In another example, HSV according to the present disclosure has at least 40% infectivity. In another example, HSV according to the present disclosure has at least 50% infectivity. In another example, HSV according to the present disclosure has between 15 and 80% infectivity. In another example, HSV according to the present disclosure has between 20 and 80% infectivity. In another example, HSV according to the present disclosure has between 30 and 80% infectivity. In another example, HSV according to the present disclosure has between 35 and 80% infectivity. In another example, HSV according to the present disclosure has between 45 and 80% infectivity. In another example, HSV according to the present disclosure has between 55 and 80% infectivity.

Viral infectivity can also be determined using various routine methods such as plaque assay (Exemplary assays described in Dulbecco and Vogt (1953) Cold Spring Harbor Symp. Quant. Biol., 18: 273-279; Johnson et al. (1990) Quantitative Assays for Virus Infectivity. In: Aldovini A., Walker B.D. (eds) Techniques in HIV Research. Palgrave Macmillan, London).

The present inventors have also found HSV expressing PTEN-alpha transgene has a high rate of replication in mesenchymal lineage precursor or stem cells. For example, HSV expressing PTEN-alpha transgene can have at least 10% increased replication relative to corresponding HSV control. In an example, HSV expressing PTEN-alpha transgene has at least 20% increased replication relative to corresponding HSV control. In another example, HSV expressing PTEN-alpha transgene has at least 30% increased replication relative to corresponding HSV control. In another example, HSV expressing PTEN-alpha transgene has between 20% and 40% increased replication relative to corresponding HSV control.

### Delivery to Cancer Cells

The present inventors have identified that mesenchymal lineage precursor or stem cells can migrate towards cancer cells and transfer a payload such as a virus or transgene expressed by the same. Accordingly, in an example, the present disclosure encompasses methods of delivering an above referenced oncolytic virus to cancer cells by administering mesenchymal lineage precursor or stem cells disclosed herein to a subject. In an example, viral payload can be transferred by contacting cancer cells with mesenchymal lineage precursor or stem cells that have been modified to introduce an above referenced oncolytic virus. For the avoidance of doubt the oncolytic virus being delivered to a cancer cell is the oncolytic virus introduced to the mesenchymal lineage precursor or stem cell. In another example, the present disclosure encompasses a method of increasing PTENα expression in a cell, the method comprising contacting the cell with a population disclosed herein. In this example, the cell can be a cancer cell. In an example, increasing PTENα expression in the cell reduces the level of phosphorylated AKT in the cell. In an example, the method is performed in vivo. For example, a population disclosed herein can be administered to a subject.

The term “contacting” is used in the context of the present disclosure to refer to “direct” or “indirect” contact. “Direct contact” is used in the context of the present disclosure to refer to physical contact between the cancer cell and a modified mesenchymal lineage precursor or stem cell that facilitates transfer of payload such as an oncolytic virus and/or transgene expressed by the same. For example, a cancer cell and a modified mesenchymal lineage precursor or stem cell can be in direct contact via a common connexin (i.e. a connexin that is expressed by both the cancer cell and the modified mesenchymal lineage precursor or stem cell). In this example, the common connexin facilitates transfer of payload from the mesenchymal lineage precursor or stem cell to the cancer cell via a gap junction.

“Indirect contact” is used in the context of the present disclosure to refer to delivery of oncolytic virus from a modified mesenchymal lineage precursor or stem cell to a cancer cell without direct contact. For example, a modified mesenchymal lineage precursor or stem cell in close proximity to a cancer cell may be in indirect contact with the cancer cell. In an example, a modified mesenchymal lineage precursor or stem cell in indirect contact with a cancer cell can deliver payload to the cancer cell via exosomes. In another example, a modified mesenchymal lineage precursor or stem cell in indirect contact with a cancer cell can deliver payload to the cancer cell via secretion into the surrounding environment.

In an example, both direct and indirect contact can be mediated by administering a population disclosed herein to a subject.

In another example, a modified mesenchymal lineage precursor or stem cell in direct contact with a cancer cell can deliver payload to the cancer cell via a common connexin and indirectly via exosomes.

Cancer cells receiving payload from a modified mesenchymal lineage precursor or stem cell are not particularly limited so long as they can be directly or indirectly contacted by the modified mesenchymal lineage precursor or stem cell to facilitate transfer of oncolytic virus. In an example, the cancer cell is a brain cancer cell. For example, the cancer cell can be from a glioblastoma. In an example, the cancer cell is a glioma cell. In an example, the cancer cell is a pancreatic cancer cell. In another example, the cancer cell is a lung cancer cell. In another example, the cancer cell is a cervical cancer cell. In another example, the cancer cell is a colorectal cancer cell. In another example, the cancer cell is a liver cancer cell. In another example, the cancer cell is an osteosarcoma cell. In another example, the cancer cell is a prostate cancer cell. In another example, the cancer cell is a melanoma cell. In an example, the cancer cell is a breast cancer cell. In an example, the cancer cell is a PTEN deficient cancer cell.

In another example, the cancer cell is a syncytial cancer cell. The term “syncytial” is used in the context of the present disclosure to refer to cancerous tissue or mass that is made up of cells interconnected by specialized membrane with gap junctions, which are synchronized electrically in an action potential.

Delivery of oncolytic virus from a modified mesenchymal lineage precursor or stem cells to a cancer cell can be facilitated in vivo via various exemplary routes. For example, mesenchymal lineage precursor or stem cells may be administered systemically, such as, for example, by intravenous, intraarterial, or intraperitoneal administration. In other examples, the mesenchymal lineage precursor or stem cells can be administered by intranasal or intramuscular administration. In an example, the mesenchymal lineage precursor or stem cells are administered to a site in close proximity to a cancer cell such as surrounding tissue. In another example, the mesenchymal lineage precursor or stem cells are administered directly into the cancer.

### Method of Treatment

In one example, cell populations and compositions comprising the same according to the present disclosure can be administered for the treatment of a cancer. The term “cancer” refers to or describes the physiological condition in mammals that is typically characterized by unregulated cell growth. Examples of cancer include but are not limited to, carcinoma, lymphoma, blastoma, sarcoma, and leukemia or lymphoid malignancies. More particular examples of such cancers include, but are not limited to, squamous cell cancer (e.g. epithelial squamous cell cancer), lung cancer including small-cell lung cancer, non-small cell lung cancer, adenocarcinoma of the lung and squamous carcinoma of the lung, cancer of the peritoneum, hepatocellular cancer, gastric or stomach cancer including gastrointestinal cancer and gastrointestinal stromal cancer, pancreatic cancer, glioblastoma, cervical cancer, ovarian cancer, liver cancer, bladder cancer, cancer of the urinary tract, hepatoma, breast cancer, colon cancer, rectal cancer, colorectal cancer, endometrial or uterine carcinoma, salivary gland carcinoma, kidney or renal cancer, prostate cancer, vulval cancer, thyroid cancer, hepatic carcinoma, anal carcinoma, penile carcinoma, melanoma, superficial spreading melanoma, lentigo maligna melanoma, acral lentiginous melanomas, nodular melanomas, multiple myeloma and B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia); chronic lymphocytic leukemia (CLL); acute lymphoblastic leukemia (ALL); hairy cell leukemia; chronic myeloblastic leukemia; and post-transplant lymphoproliferative disorder (PTLD), as well as abnormal vascular proliferation associated with phakomatoses, edema (such as that associated with brain tumors), Meigs' syndrome, brain, as well as head and neck cancer, and associated metastases.

In an example, the cancer is brain cancer. In an example, the cancer is glioblastoma. In an example, the cancer is pancreatic cancer. In another example, the cancer is lung cancer. In another example, the cancer is cervical cancer. In another example, the cancer is colorectal cancer. In another example, the cancer is liver cancer. In another example, the cancer is osteosarcoma. In another example, the cancer is prostate cancer. In another example, the cancer is melanoma.

In an example, the cancer is a PTEN mutated or deficient cancer. In an example, the cancer is a PTEN mutated or deficient glioblastoma, endometrial cancer, colon cancer, lung cancer, breast cancer, prostate cancer and ovarian cancer. In an example, the cancer is a PTEN mutated or deficient breast cancer. In another example, the cancer is as PTEN mutated or deficient brain cancer. In an example, cell populations and compositions comprising the same according to the present disclosure can be used in methods of killing cancer cells. In an example, cancer cells killed using such methods can be from the above referenced cancer types.

### Cellular Compositions

The present disclosure encompasses populations of mesenchymal precursor lineage or stem cells. Such populations can be provided in a composition. For example, in performing the methods of the present disclosure mesenchymal lineage precursor or stem cells can be provided in a composition suitable for administration to a subject.

Exemplary compositions according to the present disclosure can comprise mesenchymal lineage precursor or stem cells that have been modified to introduce HSV. Exemplary HSV are described above. In an example, compositions according to the present disclosure can comprise mesenchymal lineage precursor or stem cells modified to introduce an above referenced oncolytic virus or a combination thereof. For example, mesenchymal lineage precursor or stem cells can be modified to introduce a HSV comprising a PTEN-alpha transgene. In an example, the PTEN-alpha transgene comprises a nucleic acid sequence shown in SEQ ID NO: 1.

In another example, compositions according to the present disclosure can comprise mesenchymal lineage precursor or stem cells modified to introduce a HSV that has high level of infectivity in the mesenchymal lineage precursor or stem cells. In an example, the level of infectivity exceeds 15% of mesenchymal lineage precursor or stem cells. In another example, the level of infectivity exceeds 25% of mesenchymal lineage precursor or stem cells. In another example, the level of infectivity exceeds 35% of mesenchymal lineage precursor or stem cells. In another example, the level of infectivity exceeds 45% of mesenchymal lineage precursor or stem cells.

In another example, compositions according to the present disclosure can comprise mesenchymal lineage precursor or stem cells modified to introduce a HSV that does not substantially affect viability of the mesenchymal lineage precursor or stem cell.

In another example, compositions according to the present disclosure can comprise mesenchymal lineage precursor or stem cells modified to introduce a HSV that does not kill the mesenchymal lineage precursor or stem cells before they can deliver the oncolytic virus to a cancer cell.

In one example, such a composition comprises a pharmaceutically acceptable carrier and/or excipient.

The terms “carrier” and “excipient” refer to compositions of matter that are conventionally used in the art to facilitate the storage, administration, and/or the biological activity of an active compound (see, e.g., Remington's Pharmaceutical Sciences, 16th Ed., Mac Publishing Company (1980). A carrier may also reduce any undesirable side effects of the active compound. A suitable carrier is, for example, stable, e.g., incapable of reacting with other ingredients in the carrier. In one example, the carrier does not produce significant local or systemic adverse effect in recipients at the dosages and concentrations employed for treatment.

Suitable carriers for the present disclosure include those conventionally used, e.g., water, saline, aqueous dextrose, lactose, Ringer's solution, a buffered solution, hyaluronan and glycols are exemplary liquid carriers, particularly (when isotonic) for solutions. Suitable pharmaceutical carriers and excipients include starch, cellulose, glucose, lactose, sucrose, gelatin, malt, rice, flour, chalk, silica gel, magnesium stearate, sodium stearate, glycerol monostearate, sodium chloride, glycerol, propylene glycol, water, ethanol, and the like.

In another example, a carrier is a media composition, e.g., in which a cell is grown or suspended. Such a media composition does not induce any adverse effects in a subject to whom it is administered.

Exemplary carriers and excipients do not adversely affect the viability of a cell and/or the ability of a cell to treat or prevent disease.

In one example, the carrier or excipient provides a buffering activity to maintain the cells and/or soluble factors at a suitable pH to thereby exert a biological activity, e.g., the carrier or excipient is phosphate buffered saline (PBS). PBS represents an attractive carrier or excipient because it interacts with cells and factors minimally and permits rapid release of the cells and factors, in such a case, the composition of the disclosure may be produced as a liquid for direct application to the blood stream or into a tissue or a region surrounding or adjacent to a tissue, e.g., by injection.

The cellular compositions described herein may be administered alone or as admixtures with other cells. The cells of different types may be admixed with a composition of the disclosure immediately or shortly prior to administration, or they may be co-cultured together for a period of time prior to administration.

In one example, the composition comprises an effective amount or a therapeutically effective amount of cells. For example, the composition comprises about 1×10⁵ cells to about 1×10⁹ cells or about 1.25×10³ cells to about 1.25×10⁷ cells. The exact amount of cells to be administered is dependent upon a variety of factors, including the age, weight, and sex of the subject, and the extent and severity of the disorder being treated.

Exemplary dosages include at least about 1.2×10⁸ to about 8×10¹⁰ cells, such as between about 1.3×10⁸ to about 8×10⁹ cells, about 1.4×10⁸ to about 8×10⁸ cells, about 1.5×10⁸ to about 7.2×10⁸ cells, about 1.6×10⁸ to about 6.4×10⁸ cells, about 1.7×10⁸ to about 5.6×10⁸ cells, about 1.8×10⁸ to about 4.8×10⁸ cells, about 1.9×10⁸ to about 4.0×10⁸ cells, about 2.0×10⁸ to about 3.2×10⁸ cells, about 2.1×10⁸ to about 2.4×10⁸ cells. For example, a dose can include at least about 1.5×10⁸ cells. For example, a dose can include at least about 2.0×10⁸ cells.

Put another way, exemplary doses include at least about 1.5×10⁶ cells/kg (80kg subject). In an example, a dose can include at least about 2.5×10⁶ cells/kg. In other examples, a dose can comprise between about 1.5×10⁶ to about 1×10⁹ cells/kg, about 1.6×10⁶ to about 1×10⁸ cells/kg, about 1.8×10⁶ to about 1×10⁷ cells/kg, about 1.9×10⁶ to about 9×10⁶ cells/kg, about 2.0×10⁶ to about 8×10⁶ cells/kg, about 2.1×10⁶ to about 7×10⁶ cells/kg, about 2.3×10⁶ to about 6×10⁶ cells/kg, about 2.4×10⁶ to about 5×10⁶ cells/kg, about 2.5×10⁶ to about 4×10⁶ cells/kg, about 2.6×10⁶ to about 3×10⁶ cells/kg.

In an example, modified mesenchymal lineage precursor or stem cells comprise at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 99% of the cell population of the composition.

Compositions of the disclosure may be cryopreserved. Cryopreservation of mesenchymal lineage precursor or stem cells can be carried out using slow-rate cooling methods or ‘fast’ freezing protocols known in the art. Preferably, the method of cryopreservation maintains similar phenotypes, cell surface markers and growth rates of cryopreserved cells in comparison with unfrozen cells.

The cryopreserved composition may comprise a cryopreservation solution. The pH of the cryopreservation solution is typically 6.5 to 8, preferably 7.4.

The cyropreservation solution may comprise a sterile, non-pyrogenic isotonic solution such as, for example, PlasmaLyte A™. 100 mL of PlasmaLyte A™ contains 526 mg of sodium chloride, USP (NaCl); 502 mg of sodium gluconate (C6H11NaO7); 368 mg of sodium acetate trihydrate, USP (C2H3NaO2.3H2O); 37 mg of potassium chloride, USP (KC1); and 30 mg of magnesium chloride, USP (MgCl2.6H2O). It contains no antimicrobial agents. The pH is adjusted with sodium hydroxide. The pH is 7.4 (6.5 to 8.0).

The cryopreservation solution may comprise Profreeze™. The cryopreservation solution may additionally or alternatively comprise culture medium, for example, aMEM.

To facilitate freezing, a cryoprotectant such as, for example, dimethylsulfoxide (DMSO), is usually added to the cryopreservation solution. Ideally, the cryoprotectant should be nontoxic for cells and patients, nonantigenic, chemically inert, provide high survival rate after thawing and allow transplantation without washing. However, the most commonly used cryoprotector, DMSO, shows some cytotoxicity . Hydroxylethyl starch (HES) may be used as a substitute or in combination with DMSO to reduce cytotoxicity of the cryopreservation solution.

The cryopreservation solution may comprise one or more of DMSO, hydroxyethyl starch, human serum components and other protein bulking agents. In one example, the cryopreserved solution comprises about 5% human serum albumin (HSA) and about 10% DMSO. The cryopreservation solution may further comprise one or more of methycellulose, polyvinyl pyrrolidone (PVP) and trehalose.

In one embodiment, cells are suspended in 42.5% Profreeze^{Tm}/50% aMEM/7.5% DMSO and cooled in a controlled-rate freezer.

The cryopreserved composition may be thawed and administered directly to the subject or added to another solution, for example, comprising hyaluronic acid. Alternatively, the cryopreserved composition may be thawed and the mesenchymal lineage precursor or stem cells resuspended in an alternate carrier prior to administration.

In an example, the cellular compositions described herein may be administered as a single dose. In another example, cellular compositions are administered over multiple doses. For example, at least 2, at least 3, at least 4, at least 5, at least 6, at least 7, at least 8, at least 9, at least 10 doses.

In one example, mesenchymal lineage precursor or stem cells can be culture expanded prior to administration. Various methods of mesenchymal lineage precursor or stem cell culture are known in the art. In an example, mesenchymal lineage precursor or stem cells are culture expanded in a serum free medium prior to administration. For example, mesenchymal lineage precursor or stem cells can be passaged at least once, twice, three, four, five, six, seven, eight, nine, 10 or more times prior to administration.

Mesenchymal lineage precursor or stem cells may be administered systemically, such as, for example, by intravenous, intraarterial, or intraperitoneal administration. The mesenchymal lineage precursor or stem cells may also be administered by intranasal, intramuscular or intracardiac administration. In an example, the mesenchymal lineage precursor or stem cells are administered directly into a subject's tumour.

### EXAMPLES

### Example 1

### HSV-P10 Loading of Mesenchymal Stem Cells (MSC)

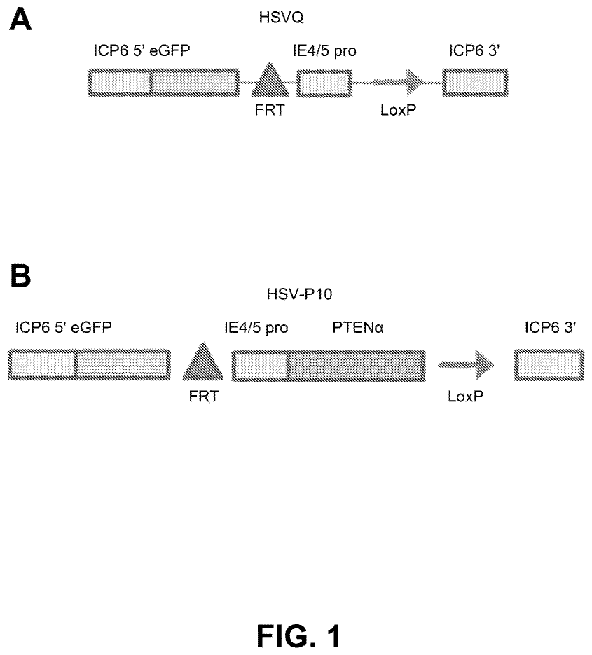

PTENα expressing herpes simplex virus (HSV-P10), an oncolytic virus, was generated using a modified PTENα gene sequence, whereby the PTENα CUG start codon is mutated to AUG to enhance translation of the full-length N-terminally extended protein, and the internal canonical PTEN AUG start codon is mutated to AUA to abrogate canonical PTEN expression from the construction. PTENα was incorporated into a oncolytic HSV1 backbone deleted for both copies of y34.5 within the ICP6 gene locus of the virus. FIG. 1 depicts the structure of the genetic manipulations engineered within the ICP6 locus in the control (HSVQ) and HSV-P10 viruses used in the study.

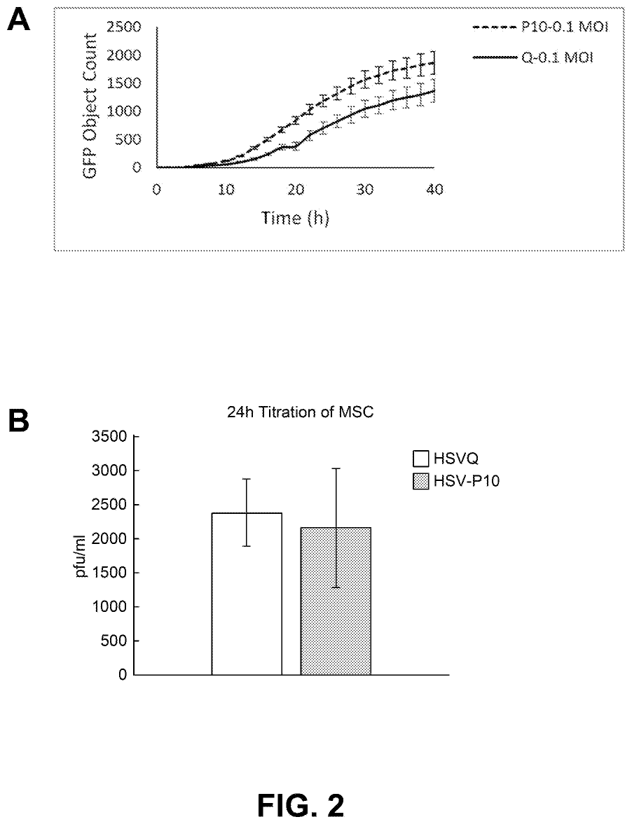

Mesenchymal stems cells were loaded with either HSVQ or HSV-P10 at multiplicity of infection (MOI) 0.025, 0.05, 0.1, 0.2 and 0.5 and infection was determined by the detection of GFP in the cells over time (FIGS. 2A and 2E). GFP was monitored over time utilizing the Cytation 5 Cell Imaging Multi-Mode Reader in conjunction with a BioSpa 8 Automated Incubator (Biotek Instruments, INC.). GFP object count was quantified and graphed as an average of 4 wells per treatment group ±SEM. The rate of replication within the cells correlated with the MOI of HSVQ or HSV-P10 used to infect the mesenchymal stems cells.

To determine the kinetics of HSV-P10 and HSVQ viral replication in mesenchymal stems cells, a comparison of HSV-P10 and HSVQ loaded mesenchymal stems cells was performed (FIG. 2A). Mesenchymal stem cells at 3×10⁶ cells were plated in 6 well plates and cultured for 24 hrs. The plated mesenchymal stem cells were infected with 1 MOI of HSVQ or HSV-P10 for 1 hr. After incubation, the media was removed and replaced with fresh DMEM and cultured for another 24 hrs. HSVQ or HSV-P10 loaded mesenchymal stem cells and conditioned media were harvested and titration studies were performed on vero cells.

HSV-P10 appeared to have superior kinetics of viral replication compared to HSVQ (FIG. 2A). However, the viral tire of HSV-P10 loaded mesenchymal stems cells was comparable to the viral tire of HSVQ loaded mesenchymal stems cells (FIG. 2B). Viral replication of HSV-P10 and HSVQ were observed in loaded mesenchymal stems cells even after 5 passages in vitro.

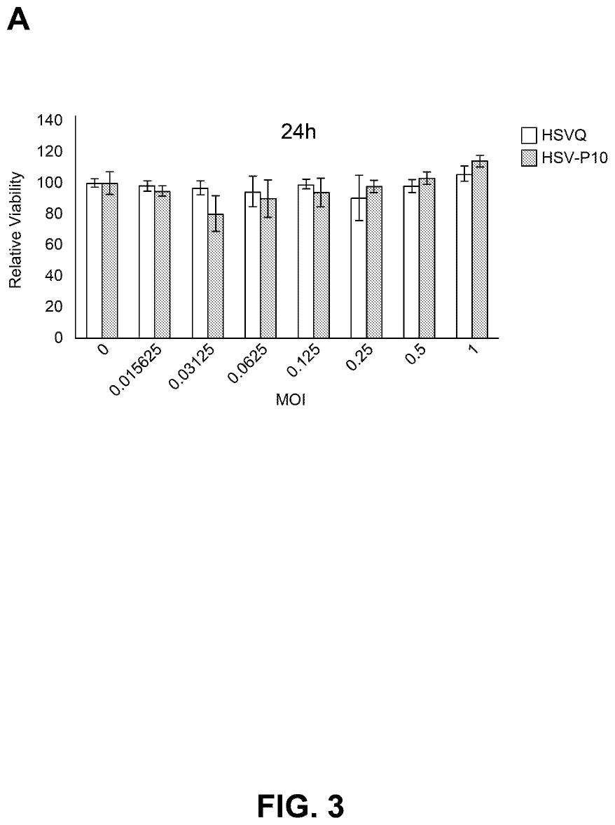

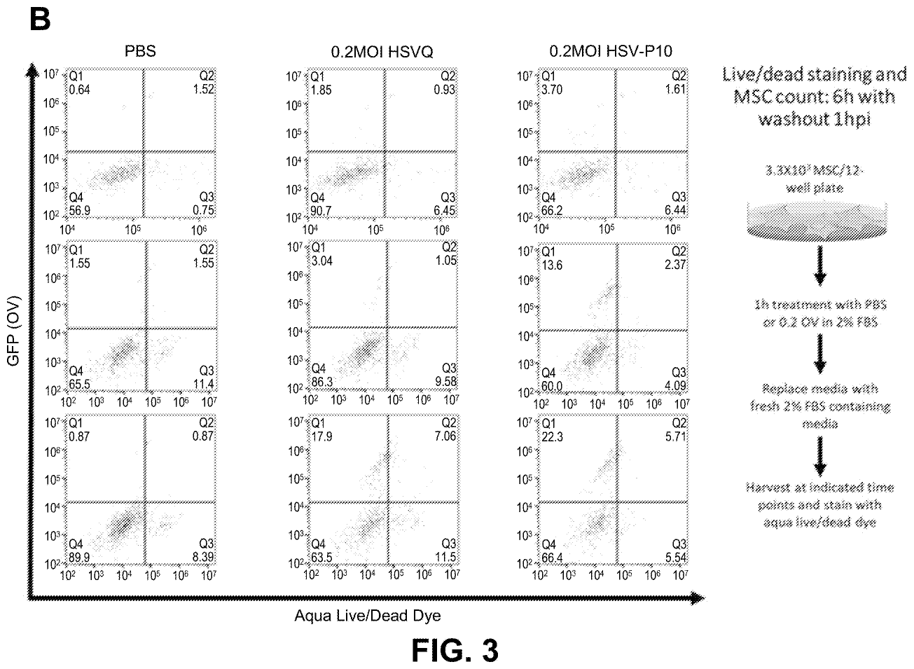

To determine the effect of viral loading on the viability of mesenchymal stems cells, cytosolic activity (aqua live/dead dye) and GFP expression was determined in loaded mesenchymal stems cells assessed by flow cytometry and quantified and represented as histograms (FIG. 3). The data demonstrates that HSV-10 and HSVQ loaded mesenchymal stems cells were viable 24 hrs post infection (FIG. 3A). Flow cytometry quadrants are shown in FIG. 3B.

### Example 2

### Evaluation of Functional PTENα Expressed by HSV-P10 Loaded Mesenchymal Stem Cells (MSC)

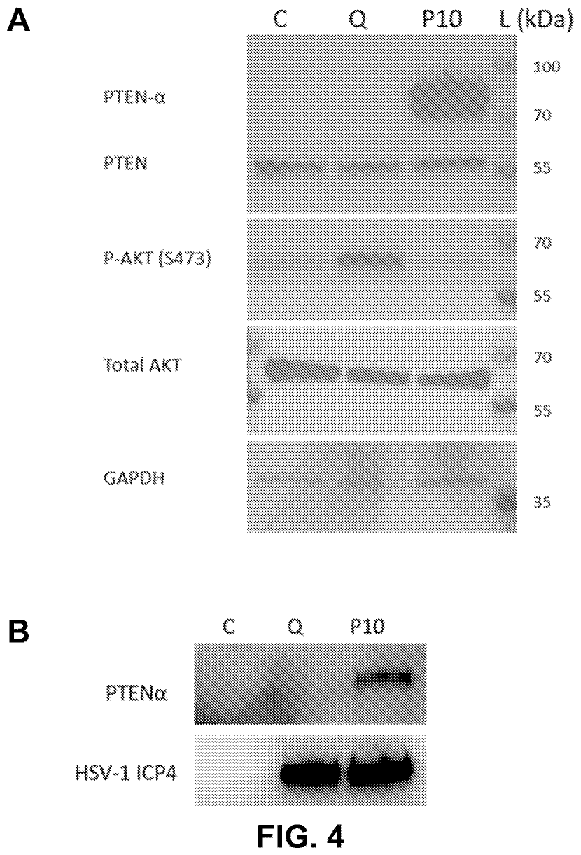

To evaluate the functionality of PTENα expressed by HSV-P10, the impact of HSV-P10 on the PI3K/AKT signalling pathway of HSV-P10 loaded mesenchymal stem cells was determined. Western blot analysis revealed an increase in AKT in HSVQ loaded mesenchymal stem cells, while HSV-P10 loaded mesenchymal stem cells which expressed PTENα had reduced phosphorylated AKT compared with control virus loading (FIG. 4A). PTENα was detected in the conditioned media of HSV-P10 loaded mesenchymal stem cells suggesting secretion of PTENα by the HSV-P10 loaded mesenchymal stem cells (FIG. 4B).

### Example 3

### Effect of HSV-P10 Loaded Mesenchymal Stem Cells on Tumour Cells

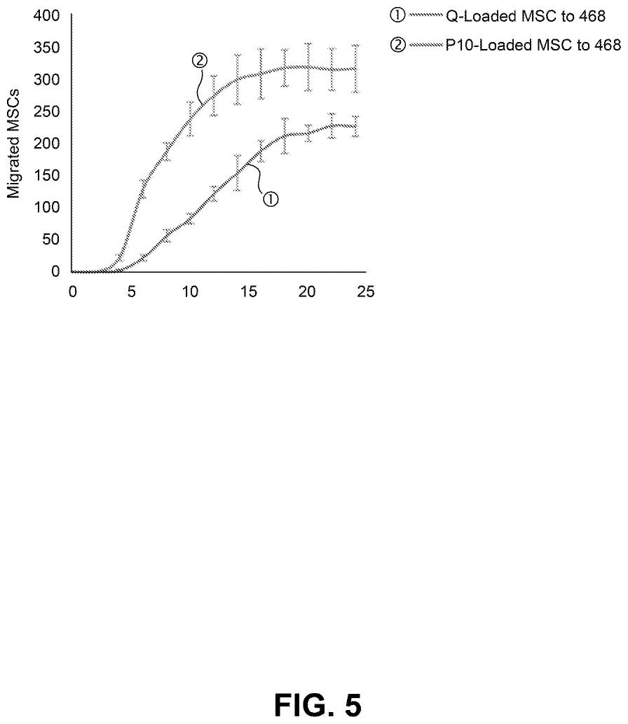

To determine the ability of HSV-P10 loaded mesenchymal stem cells to deliver the HSV-P10 to cancer cells, Boyden chamber assay was conducted and migration by monitoring viral GFP over time utilizing the Cytation 5 Cell Imaging Multi-Mode Reader in conjunction with a BioSpa 8 Automated Incubator (Biotek Instruments, INC.). However, analysis of HSVQ and HSV-P10 loaded mesenchymal stem cell migration surprisingly revealed increased kinetics of HSV-P10 loaded mesenchymal stem cells to the human breast cancer cells (MDA-468) compared to HSVQ loaded mesenchymal stem cells (FIG. 5).

### Example 4

### Effect of HSV-P10 Loaded Mesenchymal Stem Cells on Primary Human Glioma Cells

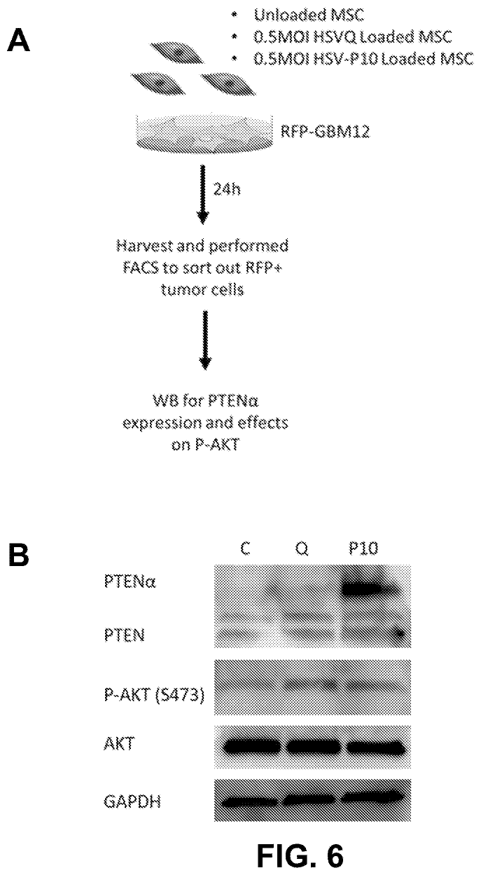

HSVQ and HSV-P10 loaded mesenchymal stem cells were co-cultured with RPF expressing GMB12 primary human glioma cells (FIG. 6A). Functionality of PTENα expressed by HSV-P10 loaded mesenchymal stem cells on the PI3K/AKT signalling pathway was determined. Western blot analysis revealed an increase PTENα and a reduction in phosphorylated AKT in glioma cells after co-culture with MSCs (FIG. 6B).

### Example 5

### Effect of HSV-P10 Loaded Mesenchymal Stem Cells on Breast Cancer Cells

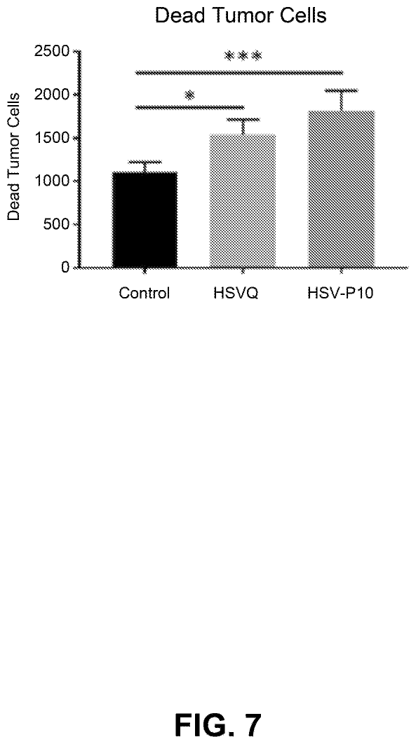

Co-culture of HSV-P10 loaded mesenchymal stem cells with DB7 murine breast cancer cells resulted in transfer of the HSV-P10 to cancer cells and induction of cell death in those cancer cells as determined by cytosolic activity (aqua live/dead dye) and GFP expression. An increase in the total amount of dead DB7 murine breast cancer cells was observed following co-culture with HSV-Q loaded mesenchymal stem cells compared to unloaded mesenchymal stem cells (control) (FIG. 7). A further increase in the total amount of dead DB7 murine breast cancer cells was observed following co-culture with HSV-P10 loaded mesenchymal stem cells compared to unloaded mesenchymal stem cells (control) and HSV-Q loaded cells (FIG. 7).

### Example 6

### Cancer Therapy

Mesenchymal lineage precursor or stem cells are loaded with a recombinant virus comprising a polynucleotide encoding Phosphatase and Tensin Homolog deleted on chromosome 10 alpha (PTENα) before being administered to a subject diagnosed with a PTEN mutated or deficient cancer. About 200 million loaded mesenchymal lineage precursor cells are administered to the subject.

Treated subjects are evaluated for safety and efficacy of therapy over about 2-6 weeks. Further doses of loaded mesenchymal lineage precursor or stem cells are administered as required.

### Example 7

### Cancer Therapy

Mesenchymal lineage precursor or stem cells are loaded with a recombinant virus comprising a herpes simplex virus (HSV) backbone and a polynucleotide encoding Phosphatase and Tensin Homolog deleted on chromosome 10 alpha (PTENα) before being administered to a subject diagnosed with cancer. Mesenchymal lineage precursor or stem cells are loaded with about 10-50 infectious units (i.u.)/MPC by addition of virus to the mesenchymal lineage precursor or stem cell culture medium. About 200 million loaded mesenchymal lineage precursor or stem cells are administered to the subject.

Treated subjects are evaluated for safety and efficacy of therapy over about 2-6 weeks. Further doses of loaded mesenchymal lineage precursor or stem cells are administered as required.

It will be appreciated by persons skilled in the art that numerous variations and/or modifications may be made to the disclosure as shown in the specific embodiments without departing from the spirit or scope of the disclosure as broadly described. The present embodiments are, therefore, to be considered in all respects as illustrative and not restrictive.

All publications discussed above are incorporated herein in their entirety.

This application claims priority from 62/882840 filed on 5 Aug. 2019 the disclosures of which are incorporated herein in their entirety.

Any discussion of documents, acts, materials, devices, articles or the like which has been included in the present specification is solely for the purpose of providing a context for the present disclosure. It is not to be taken as an admission that any or all of these matters form part of the prior art base or were common general knowledge in the field relevant to the present disclosure as it existed before the priority date of each claim of this application.

### REFERENCES

Ausubel et al. (editors) (1988, including all updates until present) Current Protocols in Molecular Biology, Greene Pub. Associates and Wiley-Interscience.
Bader et al. (2011) Gene Ther. 18:1121-6.
Brown T A (editor) (1991) Essential Molecular Biology: A Practical Approach, Volumes 1 and 2, IRL Press.
Coligan et al. (editors) (including all updates until present) Current Protocols in Immunology, John Wiley & Sons.
Glover and Hames (editors) (1995 & 1996) DNA Cloning: A Practical Approach, Volumes 1-4, IRL Press.
Griffiths-Jones, S. 2004 Nucl Acids Res, 32, D109-D111.
Harlow and Lane (editors) (1988) Antibodies: A Laboratory Manual, Cold Spring Harbour Laboratory.
Kozomara et al. 2013; Nucl Acids Res, 42, D68-D73.
Lennox and Behlke (2011) Gene Ther. 18″1111-20.
Perbal J (1984) A Practical Guide to Molecular Cloning, John Wiley and Sons.
Sambrook et al., (1989) Molecular Cloning: A Laboratory Manual, Cold Spring Harbour Laboratory Press.
Simmons & Torok-Storb (1991) Blood. 78:55-62.

1. A population of mesenchymal lineage precursor or stem cells, wherein said cells are modified to increase expression of Phosphatase and Tensin Homolog deleted on chromosome 10 alpha (PTENα).

2. The population of claim 1, wherein the increase in expression of PTENα is sufficient to decrease the level of phos phorylated AKT in modified cells.

3. The population of claim 1 or claim 2, wherein the increase in expression of PTENα is sufficient to enhance killing of tumour cells.

4. The population according to any one of claims 1 to 3, wherein the increase in expression of PTENα is sufficient to enhance migration to tumour cells.

5. 5.The population according to any one of claims 1 to 4, wherein the mesenchymal lineage precursor or stem cells are modified to introduce a recombinant virus which comprises a polynucleotide encoding PTENα.

6. The population of claim 5, wherein the recombinant virus is an oncolytic virus. The population of claim 5 or claim 6, wherein the virus comprises a herpes simplex virus (HSV) backbone.

8. The population according to any one of claims 4 to 7, wherein between 20% and 80% of cells comprise the recombinant virus.

9. The population according to any one of claims 4 to 8, wherein the polynucleotide encoding PTENα is operatively linked to a tumour specific promoter or an inducible promoter.

10. The population of claim 9, wherein the tumour specific promoter is a survivin promoter, COX-2 promoter, PSA promoter, CXCR4 promoter, STAT3 promoter, hTERT promoter, AFP promoter, CCKAR promoter, CEA promoter, erbB2 promoter, E2F1 promoter, HE4 promoter, LP promoter, MUC-1 promoter, TRP1 promoter, Tyr promoter.

11. The population according to any one of claims 4 to 10, wherein the recombinant virus comprises a capsid protein that binds a tumour-specific cell surface molecule.

12. The population of claim 11, wherein the capsid protein is a fibre, a penton or hexon protein.

13. The population according to any one of claims 4 to 12, wherein the recombinant virus comprises a nucleic acid sequence as shown in SEQ ID NO: 1.

14. The population according to any one of claims 4 to 13, wherein the recombinant virus is a HSV.

15. The population according to any one of claims 3 to 14, wherein the tumour cells are breast cancer or brain cancer cells.

16. The population according to any one of claims 1 to 15, wherein the mesenchymal lineage precursor or stem cells are MSCs.

17. The population according to any one of claims 1 to 16, wherein the mesenchymal lineage precursor or stem cells have been purified by immunoselection.

18. The population according to any one of claims 1 to 15, wherein the mesenchymal lineage precursor or stem cell expresses STRO-1.

19. The population according to any one of claims 1 to 18, wherein the mesenchymal lineage precursor or stem cells are derived from pluripotent cells.

20. The population of claim 19, wherein the pluripotent cells are induced pluripotent stem (iPS) cells.

21. The population according to any one of claims 1 to 20, wherein the population of cells has been culture expanded.

22. A method of increasing PTENα expression in a cell, the method comprising contacting the cell with a population according to any one of claims 1-21.

23. The method of claim 22, wherein the contacted cell is a cancer cell.

24. The method of claim 22 or claim 23, wherein increasing PTENα expression in the cell reduces the level of phosphorylated AKT in the cell.

25. The method according to any one of claims 22 to 24, wherein the method is performed in-vivo.

26. A pharmaceutical composition comprising a population according to any one of claims 1 to 21.

27. A method of treating cancer in a subject, the method comprising administering a population according to any one of claims 1 to 21 or the composition of claim 26.

28. A method of killing cancer cells, the method comprising contacting a population of cancer cells with a population according to any one of claims 1 to 21 or the composition of claim 26.

29. A method of delivering mesenchymal lineage precursor or stem cells to cancer cells in a subject, the method comprising administering a population according to any one of claims 1 to 21 or the composition of claim 26.

30. The method according to any one of claims 27 to 29, wherein the cancer is selected from the group consisting of lung cancer, pancreatic cancer, colorectal cancer, liver cancer, cervical cancer, prostate cancer, breast cancer, endometrial cancer, thyroid cancer, kidney cancer, brain cancer, glioblastoma, osteosarcoma and melanoma.

31. The method or claim 30, wherein the cancer is breast cancer or brain cancer.

32. The method according to any one of claim 27, 29 or 30, wherein the population or composition is administered to the subject by intravenous, intra-arterial, intra-tumoral or intraperitoneal administration.

33. Use of a population according to any one of claims 1 to 21 in the manufacture of a medicament for treating cancer.

34. Use of a population according to any one of claims 1 to 21 in the manufacture of a medicament for delivering mesenchymal lineage precursor or stem cells to cancer cells.


SEQUENCE LISTING (2 sequences)

SEQ ID NO: 1 - DNA, 1200 bp, Homo sapiens
atggagcggggaggagaagcggcggcggcggcggccgcggcggctgcagctccagggagggggtctgagtcgcctgtcaccatttccagggctgggaacgccggagagttggtctctccccttctactgcctccaacacggcggcggcggcggcggcacatccagggacccgggccggttttaaacctcccgtccgccgccgccgcaccccccgtggcccgggctccggaggccgccggcggaggcagccgttcggaggattattcgtcttctccccattccgctgccgccgctgccaggcctctggctgctgaggagaagcaggcccagtcgctgcaaccatccagcagccgccgcagcagccattacccggctgcggtccagagccaagcggcggcagagcgaggggcatcagctaccgccaagtccagagccatttccatcctgcagaagaagccccgccaccagcagcttctgccatctctctcctcctttttcttcagccacaggctcccagacataacagccatcatcaaagagatcgttagcagaaacaaaaggagatatcaagaggatggattcgacttagacttgacctatatttatccaaacattattgctatgggatttcctgcagaaagacttgaaggcgtatacaggaacaatattgatgatgtagtaaggtttttggattcaaagcataaaaaccattacaagatatacaatctttgtgctgaaagacattatgacaccgccaaatttaattgcagagttgcacaatatccttttgaagaccataacccaccacagctagaacttatcaaacccttttgtgaagatcttgaccaatggctaagtgaagatgacaatcatgttgcagcaattcactgtaaagctggaaagggacgaactggtgtaatgatatgtgcatatttattacatcggggcaaatttttaaaggcacaagaggccctagatttctatggggaagtaaggaccagagacaaaaagggagtaactattcccagtcagaggcgctatgtgtattattatagctacctgttaaagaatcatctggattatagaccagtggcactgttgtttcacaagatgatgtttgaaactattccaatgttcagtggcggaacttgcaatcctcagtttgtggtctgccagctaaaggtgaagatatattcctcc

SEQ ID NO: 2 - protein, 576 aa, Homo sapiens
MERGGEAAAAAAAAAAAPGRGSESPVTISRAGNAGELVSPLLLPPTRRRRRRHIQGPGPVLNLPSAAAAPPVARAPEAAGGGSRSEDYSSSPHSAAAAARPLAAEEKQAQSLQPSSSRRSSHYPAAVQSQAAAERGASATAKSRAISILQKKPRHQQLLPSLSSFFFSHRLPDMTAIIKEIVSRNKRRYQEDGFDLDLTYIYPNIIAMGFPAERLEGVYRNNIDDVVRFLDSKHKNHYKIYNLCAERHYDTAKFNCRVAQYPFEDHNPPQLELIKPFCEDLDQWLSEDDNHVAAIHCKAGKGRTGVMICAYLLHRGKFLKAQEALDFYGEVRTRDKKGVTIPSQRRYVYYYSYLLKNHLDYRPVALLFHKMMFETIPMFSGGTCNPQFVVCQLKVKIYSSNSGPTRREDKFMYFEFPQPLPVCGDIKVEFFHKQNKMLKKDKMFHFWVNTFFIPGPEETSEKVENGSLCDQEIDSICSIERADNDKEYLVLTLTKNDLDKANKDKANRYFSPNFKVKLYFTKTVEEPSNPEASSSTSVTPDVSDNEPDHYRYSDTTDSDPENEPFDEDQHTQITKV